SEQUENCE LISTING (4 sequences)

SEQ ID NO: 1 - DNA, 23 bp, synthetic construct
  source 1..23: mol_type = other DNA
tccaaaatgtccgacaaaacagt

SEQ ID NO: 2 - DNA, 21 bp, synthetic construct
  source 1..21: mol_type = other DNA
tgcatttcagtgcatgtgtct

SEQ ID NO: 3 - DNA, 23 bp, synthetic construct
  source 1..23: mol_type = other DNA
gtctcagaaagtggaagaacagc

SEQ ID NO: 4 - DNA, 21 bp, synthetic construct
  source 1..21: mol_type = other DNA
ccaaggccagtaataccagca

(19) United States
(12) Patent Application Publication
(10) Pub. No.: US 2025/0067739 A1
(43) Pub. Date: Feb. 27, 2025

(54) METHODS OF DETECTING VIRAL OR BACTERIAL INFECTIONS

(71) Applicant: Yale University, New Haven, CT (US)

(72) Inventor: Ellen Foxman, New Haven, CT (US)

(73) Assignee: Yale University, New Haven, CT (US)

(21) Appl. No.: 18/721,445

(22) PCT Filed: Dec. 22, 2022
(86) PCT No.: PCT/US22/82314

§ 371 Date: Jun. 18, 2024

Related U.S. Application Data
(60) Provisional application No. 63/293,386, filed on Dec. 23, 2021.

(51) Int. Cl. G01N 33/569

(52) U.S. Cl. CPC G01N 33/56983; G01N 33/56911; G01N 2333/11; G01N 2333/165; G01N 2333/195

(57) ABSTRACT

In one aspect, the invention provides a method for detecting a viral-only or a bacterial-associated respiratory infection in a patient, the method comprising analyzing a respiratory sample to determine levels of at least two respiratory virus infection-associated molecules, at least two bacterial respiratory infection-associated molecules, and comparing the levels of the respiratory virus infection-associated molecules and/or the levels of the bacterial respiratory infection-associated molecules with a predetermined reference level.

### CROSS-REFERENCE TO RELATED APPLICATIONS

The present application claims priority under 35 U.S.C. § 119(e) to U.S. Provisional Patent Application No. 63/293,386, filed Dec. 23, 2021, which application is incorporated herein by reference in its entirety.

### STATEMENT REGARDING FEDERALLY SPONSORED RESEARCH OR DEVELOPMENT

This invention was made with government support under Grant Number: R21 AI156208 awarded by National Institutes of Health. The government has certain rights in the invention.

### SEQUENCE LISTING

The present application contains a Sequence Listing which has been submitted in XML format via Patent Center and is hereby incorporated by reference in its entirety. Said XML file, which was created on Dec. 22, 2022, is named 047162_7349WO1_SequenceListingST_26.xml and is 4,670 bytes in size.

### BACKGROUND OF THE INVENTION

In the past two decades, in addition to the expected annual viral infections, several emerging respiratory viruses have had a global impact including the SARS coronavirus, the 2009 swine flu, and currently the emerging 2019-coronavirus (2019-nCoV).

The COVID-19 pandemic has triggered discussions about how to expand surveillance for unrecognized or emerging pathogen. For respiratory viruses, proposed surveillance approaches include isolation of viruses from animal sources, identification of unexpected viruses in pooled human respiratory samples, and surveillance for outbreaks, as in the unexplained pneumonia surveillance project which led to the initial identification of SARS-CoV-2. These methods can be coupled with metagenomic sequencing for viral identification and molecular epidemiology. However, while screening animal or pooled human samples may identify unknown viruses, this approach does not specifically identify viruses capable of causing human disease. Monitoring for unexplained outbreaks does target human pathogens, but may find emerging viruses too late, after epidemic spread has already begun.

Current diagnostic tests for respiratory viruses detect known viruses, but do not detect unexpected viruses. Accordingly, there is an unmet need to screen for potential emerging viral pathogens to enable better preparation for future epidemic viral outbreaks.

In addition, respiratory infections are among the most common cause of physician visits. Due to the large number of viruses and bacteria that can cause these infections, it is cost-prohibitive to perform specific tests for each infection. Often the only actionable decision is whether or not the patient has a viral infection only or a bacterial infection or viral/bacterial co-infection, since the latter cases may require antibiotics. However, there is currently no simple test that can distinguish viral-only from bacteria-associated infections. Therefore, there is a need to increase the scope and efficiency of tests to distinguish whether the cause of respiratory symptoms is a viral or bacterial infection or co-infection in such clinical samples. The present invention addresses those needs.

### SUMMARY OF THE INVENTION

In one aspect, the invention provides a method for detecting and distinguishing between a viral-only or a bacterial-associated respiratory infection in a patient, the method comprising analyzing a respiratory sample to determine levels of at least two respiratory virus infection-associated molecules, at least two bacterial respiratory infection-associated molecules, or a combination thereof, comparing the levels of the respiratory virus infection-associated molecules and/or the levels of the bacterial respiratory infection-associated molecules with a predetermined reference level for the respiratory virus infection-associated molecules and/or a predetermined reference level for the bacterial respiratory infection-associated molecules; and determining if the patient has a virus-associated respiratory infection or a bacterial-associated respiratory infection based upon the comparing of the levels.

In some embodiments, the at least two respiratory virus infection-associated molecules are selected from the group comprising CXCL10, TRAIL, IL-23, CCL2, IL-10, IL-6, CCL15, TNFα, M-CSF, CX3CL1, CXCL9, IL-15, CCL22, IL-16, G-CSF, IL-la, IL-8, CCL8, BCA1, IL-1β, IFNγ, CCL17, IL-12p40, sCD40L, and CCL27. In some embodiments, the at least two respiratory virus infection-associated molecules are selected from the group comprising CXCL10, TRAIL, IL-23, CCL2, IL-10, IL-6, CCL15, M-CSF, CX3CL1, CXCL9, IL-15, CCL22, IL-16, IL-1α, CCL8, BCA1, IFNγ, CCL17, sCD40L, and CCL27. In some embodiments, the at least two respiratory virus infection-associated molecules are selected from the group comprising BCA1, IL-15, IL-10, CCL8, CCL2, CXCL10, CXCL9, TRAIL, IL-8, IL-1β, IFNγ, CCL17, IL-12p40, sCD40L, M-CSF, and/or CCL27. In some embodiments, the at least two respiratory virus infection-associated molecules are selected from the group comprising CCL8, IL-15, CXC13, IL-10, CCL2, CXCL10, TRAIL, CXCL9, IL-1β and IL-8. In some embodiments, the at least two respiratory virus infection-associated molecules are selected from the group comprising CXCL10, CCL2, and IL-10. In some embodiments, the at least two respiratory virus infection-associated molecules include CXCL10 and CCL2.

In some embodiments, the at least two bacterial respiratory infection-associated molecules are selected from the group comprising CCL5, IL-1RA, CCL11, IL-12p40, CCL3, G-CSF, IL-8, TNFα, IL-1β, CCL4, IL-1α, IL-22, IL-6, RANTES, MIP-1β, MIP-1α, Eotaxin, GROα, CCL27, MCP-3, SCF, IL-13, IL-16, IL-10, EGF, CCL17, CXCL9, and FGF-2. In some embodiments, the at least two bacterial respiratory infection-associated molecules are selected from the group comprising CCL5, IL-1RA, CCL11, IL-12p40, CCL3, G-CSF, IL-8, TNFα, IL-1β, CCL4, IL-1α, IL-22, and IL-6. In some embodiments, the at least two bacterial respiratory infection-associated molecules are selected from the group comprising CCL5, IL-1RA, CCL11, IL-12p40, CCL3, G-CSF, IL-8, TNFα, IL-1β, and CCL4. In some embodiments, the at least two bacterial respiratory infection-associated molecules are selected from the group comprising TNF, IL-8, and IL-1β.

In some embodiments, analyzing a respiratory sample comprises determining levels of CXCL10, CCL2, IL-10, IL-8, TNF, and IL-1β. In some embodiments, the expression level of the respiratory virus infection-associated molecules or the bacterial respiratory infection-associated molecules is determined by measuring the protein level of the molecule. In some embodiments, the protein level is determined by ELISA, an immunoassay, or mass spectrometry. In some embodiments, determining that the patient has a virus-associated respiratory infection includes determining that the levels of the respiratory virus infection-associated molecules are above the respective reference level. In some embodiments, the method further comprises the step of treating the patient with antiviral drugs. In some embodiments, determining that the patient has a bacterial-associated respiratory infection includes determining that the levels of the bacterial respiratory infection-associated molecules are above the respective reference level. In some embodiments, the method further comprises the step of treating the patient with antibiotics.

In another aspect, provided herein is a method of determining whether a subject who tests positive for the presence of bacterial or viral respiratory pathogen is a carrier or if the pathogen is part of the disease process, the method comprising a) analyzing a respiratory sample to determine a level of at least one respiratory virus infection-associated molecule and a level of at least one bacterial respiratory infection-associated molecule; and b) comparing the level of the at least one respiratory virus infection-associated molecule and the level of the at least one bacterial respiratory infection-associated molecule with a predetermined reference level for the at least one respiratory virus infection-associated molecule and a predetermined reference level of the at least one bacterial respiratory infection-associated molecule; wherein if the level of the at least one respiratory virus infection-associated molecule is above the respective reference level, the patient is determined to have a respiratory viral infection; if the level of the at least one bacterial respiratory infection-associated molecule is above the respective reference level, the patient is determined to have a bacterial-associated respiratory infection; if the level of the at least one respiratory virus infection-associated molecule is above the respective reference level and the level of the at least one bacterial respiratory infection-associated molecule is above the respective reference level, the patient is determined to have both a respiratory viral infection and a bacterial respiratory infection; or if neither the level of the at least one respiratory virus infection-associated molecule nor the level of the at least one bacterial respiratory infection-associated molecule is above the respective reference level, the subject is determined to be a carrier.

In another aspect, provided herein is a method for excluding the presence of a coronavirus in a sample from a patient, the method comprising a) analyzing a respiratory sample to determine an expression level of at least one respiratory virus infection-associated molecule; and b) comparing the level of the at least one respiratory virus infection-associated molecule with a predetermined reference level for the at least one respiratory virus infection-associated molecule; wherein if the level of the at least one respiratory virus infection-associated molecule is below the respective reference level, the presence of a coronavirus is excluded.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1D depict a host-response based screen for undiagnosed infections, week 4, January 2017. FIG. 1A depicts a schematic of the discovery pipeline for undiagnosed viral infections. NP samples testing negative for common respiratory viruses by PCR were screened by ELISA for CXCL10, followed by unsupervised detection of pathogen RNA using RNAseq in CXCL10-high samples. FIG. 1B represents a flow chart depicting each step of the screening process for the 359 samples tested with the Yale-New Haven hospital respiratory virus PCR panel during week 4 of January 2017. PCR panel negative, CXCL10 high samples were tested for common respiratory viruses not on the panel, including four seasonal CoV and PIV4. For virus-negative, screen positive samples, those with the highest CXCL10 concentrations were selected for RNASeq. FIG. 1C reflects the number of reads in sample A mapping to NCBI reference sequences for each of the seven gene segments of influenza C viral genome. FIG. 1D depicts the maximum likelihood phylogenetic tree of Influenza C viruses, plotted with the python package baltic 0.1.6. The tree was mid-rooted for clarity. The HEF gene sequence places this ICV isolate in the lineage Sao Paulo/82 (upper panel). This ICV clusters with ICVs circulating in Hong Kong and Japan from 2014-2018 (lower panel, zoomed version of the clade shown in the upper panel).

FIGS. 2A-2D depict the discovery of four undiagnosed cases of SARS-CoV-2 in Connecticut, March 2020. FIG. 2A depicts the number of positive tests for SARS-CoV-2 per day in the U.S, New York State, Connecticut, and Yale-New Haven Hospital. Testing began at YNHH on Mar. 13, 2020. Bracket indicates time window during which screen was performed. FIG. 2B depicts the screen for undiagnosed SARS-CoV-2 infection using PCR and CXCL10-based screening. FIG. 2C shows that of the samples collected for respiratory virus testing between 3/3-3/14/2020 (n=642), all samples with no virus detected on a 15-respiratory virus PCR panel (n=376) were screened for CXCL10 by ELISA and for SARS-CoV-2 by RT-qPCR. FIG. 2D depicts the maximum likelihood phylogenetic tree highlighting the evolutionary history of four SARS-CoV-2 isolates identified in this screen (left panel), and to different lineages and sub-lineages of local and international origins 82 (right panel, zoom of left panel. This tree was rooted at the MRCA of two early isolates from Wuhan: Wuhan/Hu-1/2019 and Wuhan/WH01/2019. The phylogeny was plotted using the python package baltic 0.1.6.

FIGS. 3A-3D depict NP immunophenotypes and relationship to NP microbes and patient age based on host, viral, and bacterial transcriptomics in discovered and control virus-positive and virus-negative samples. FIG. 3A depicts a Heatmap showing top 2678 genes across NP sample groups with Qlucore Omics explorer. Top GO biological functions for each gene cluster are indicated. The lower panel shows the relative expression of leukocyte cell-type specific differentially expressed transcripts in this dataset. FIG. 3B depicts a UMAP plot showing the relationship among transcriptomes of 55 sequenced known virus-positive or virus-negative control samples and samples discovered in the 2017 screen A-H, colored by virus. FIG. 3C depicts a UMAP plot indicating samples with >105 reads per million and >1% genome coverage for pathobionts H. influenzae or M. catarrhalis (green) >104 reads per million (yellow), or <104 reads per million (grey). FIG. 3D depicts a UMAP plot indicating samples from patients less than 5 years of age.

FIGS. 4A-4I depict proteomic signatures of antiviral and heightened innate immunity phenotypes associated with bacterial infection or viral/bacterial co-infection. and top classifiers determined by machine learning. FIG. 4A depict the top differentially expressed proteins in pairwise comparison of virus-positive and virus-negative samples (p value (p<0.05). Differentially expressed proteins were identified using Qlucore Omics explorer. For a-c, Z score represents SD from the mean. Viral load is shown as min to max Cycle threshold value by PCR (red=low Ct, high viral load, green=high Ct, low viral load; range a, Ct 34.5-11.6; b, Ct 37.2-11.6, c, Ct 30.4-13.4). FIG. 4B depict the top differentially expressed proteins in a multi-group comparison of sample groups from COVID-19, peak, COVID-19, end (as defined in text), and virus-negative controls (p<0.05). FIG. 4C depict the top differentially expressed proteins in samples with >105 reads per million from pathobionts H influenza/M. catarrhalis (red) compared to samples with <104 reads per million (grey) (p<0.01). FIG. 4E depicts accuracies of 2-feature random forest models using cytokine pairs to predict virus-positive status in proteomics data set. FIG. 4E depicts a three-dimensional plot showing log values of top performing cytokines from two-feature model in second sample set. Heatmaps show sensitivity and accuracy of paired cytokine models for predicting respiratory virus detection in this data set. FIG. 4F show the accuracies of 2-feature random forest models using cytokine pairs to predict pathobiont-high status in proteomics data set. For FIGS. 4C, 4D and 4F numeric values for sensitivity and accuracy and standard deviations are shown in Tables 10-13. FIGS. 4G-4I depict UMAP plots representing relationship among sample immunophenotypes based on Log cytokine values for top 6 cytokines identified in paired random forest models. Plots are colored by virus detected (FIG. 4G), pathobiont levels (FIG. 4H), and patient age (FIG. 4I). Ovals highlight immunophenotypes associated with virus-negative and COVID-end samples (1); COVID-peak samples (subset) (2); virus-positive samples including COVID-peak samples (subset) (3); pathobiont-high samples (4).

FIG. 5A-5B (related to FIG. 1) depict microscopic images of primary human nasal epithelial cells 7 days post-inoculation with sample A (154) into conventionally-cultured primary human nasal airway epithelial cells. FIG. 5A, Mock, day 7; FIG. 5B, plus sample A, day 7. ICV was detected in the supernatant of the culture shown in micrograph B by PCR at day 7 (not shown). Scale bar=200 microns.

FIG. 6A-6C depict transcriptional responses to viral infection with and without bacterial pathobionts. FIG. 6A depicts top upstream regulators of NP transcripts expressed in virus-positive subjects compared to negative controls, for pairwise comparisons of the following with virus-negative controls: rhinovirus-positive, all (RV), SARS-CoV-2 positive (CoV2), CoV-NL63 positive (NL63), and rhinovirus-positive, pathobiont low (RV-lo), based on ingenuity pathway analysis. Red color indicates IPA Z-score (min to max scaling, range Z=9.5-6.6). FIG. 6B depicts transcription factor binding tracks enriched in host mRNAs enriched in RV-pathobiont high compared to RV-pathobiont low NP swabs, determined by iRegulon. Red text highlights leukocyte-specific transcription factors. FIG. 6C depicts a graphical summary of ingenuity pathways differentially enriched in RV-pathobiont high compared to RV-pathobiont low NP swabs, based on 3066 differentially-expressed transcripts by DESeq (padj=0.05, FC=2.)

FIG. 7A-B depict Shapley plots showing predictive value of individual cytokines for virus positive (FIG. 7A) or pathobiont-high (FIG. 7B) status.

FIG. 8 (related to FIG. 4) depicts the relationship of NP 6-cytokine signature to sample immunophenotype, bacterial pathobiont status, and viral load.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The terms “antimicrobial” or “antimicrobial agent” mean any compound with bactericidal or bacteriostatic activity which may be used for the treatment of bacterial infection. Non-limiting examples include antibiotics.

“Biological sample” or “sample” as used herein means a biological material isolated from an individual. The biological sample may contain any biological material suitable for detecting the desired biomarkers, and may comprise cellular and/or non-cellular material obtained from the individual. A biological sample may be of any biological tissue or fluid. Frequently the sample will be a “clinical sample” which is a sample derived from a patient. Typical clinical samples include, but are not limited to, bodily fluid samples such as synovial fluid, sputum, blood, urine, blood plasma, blood serum, sweat, mucous, saliva, lymph, bronchial aspirates, peritoneal fluid, cerebrospinal fluid, and pleural fluid, and tissues samples such as blood-cells (e.g., white cells), tissue or fine needle biopsy samples and abscesses or cells therefrom. Biological samples may also include sections of tissues, such as frozen sections or formalin fixed sections taken for histological purposes.

The terms “biomarker” or “marker,” as used herein, refers to a molecule that can be detected. Therefore, a biomarker according to the present invention includes, but is not limited to, a nucleic acid, a polypeptide, a carbohydrate, a lipid, an inorganic molecule, an organic molecule, each of which may vary widely in size and properties. A “biomarker” can be a bodily substance relating to a bodily condition or disease. A “biomarker” can be detected using any means known in the art or by a previously unknown means that only becomes apparent upon consideration of the marker by the skilled artisan.

The term “biomarker (or marker) expression” as used herein, encompasses the transcription, translation, post-translation modification, and phenotypic manifestation of a gene, including all aspects of the transformation of information encoded in a gene into RNA or protein. By way of non-limiting example, marker expression includes transcription into messenger RNA (mRNA) and translation into protein. Measuring a biomarker also includes reverse transcription of RNA into cDNA (i.e. for reverse transcription-qPCR measurement of RNA levels.).

As used herein, “biomarker” in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Biomarkers can also include mutated proteins or mutated nucleic acids. Biomarkers also encompass non-blood borne factors or non-analyte physiological markers of health status, such as clinical parameters, as well as traditional laboratory risk factors. As defined by the Food and Drug Administration (FDA), a biomarker is a characteristic (e.g. measurable DNA and/or RNA) that is “objectively measured and evaluated as an indicator of normal biologic processes, pathogenic processes, or pharmacologic responses to a therapeutic intervention or other interventions”. Biomarkers also include any calculated indices created mathematically or combinations of any one or more of the foregoing measurements, including temporal trends and differences.

As used herein, the term “carrier” means a subject having viral or bacterial respiratory pathogens, i.e. virus or bacteria, in their respiratory tract but in whom these pathogens are not currently causing disease. The carrier may or may not be contagious with respect to the respiratory pathogens carried.

The term “housekeeping gene” refers to a gene where it is practical to normalize the level of other genes against the level of expression of the housekeeping gene in order to control for variables such as, but not limited to, the total amount of biological material in the sample. β-actin is one possible example of a housekeeping gene.

By the phrase “determining the level of expression” is meant an assessment of the absolute or relative quantity of a biomarker in a sample at the nucleic acid or protein level, using technology available to the skilled artisan to detect a sufficient portion of any marker.

As used herein, the term “common virus” refers to a virus that is widely known and commonly identified in a clinical setting and which a clinical investigator would expect to find in a patient based on the symptoms with which the patient presents and the clinical context. As a non-limiting example, if a patient presents at the height of influenza season and presents with symptoms consistent with those caused by the influenza virus, then influenza is a common virus in this clinical situation. As an additional non-limiting example, the viruses that a hospital sees frequently in the community that the hospital serves are common viruses. In various embodiments, the expected viruses are common cold viruses.

As used herein the term “cleared the infection” refers to a phase following the contraction of a viral infection wherein the patient's immune system has been able to successfully combat the viral infection and the patient no longer suffers from the symptoms typically associated with a viral infection as referred to above.

As used herein, an “immunoassay” refers to a biochemical test that measures the presence or concentration of a substance in a sample, such as a biological sample, using the reaction of an antibody to its cognate antigen, for example the specific binding of an antibody to a protein. Both the presence of the antigen or the amount of the antigen present can be measured.

The term “viral respiratory infection” as used herein means a virus that can cause or does cause a respiratory virus infection in a patient.

The term “bacterial respiratory infection” as used herein means a respiratory infection where the pathology is driven by bacteria.

The term “a bacterial-associated respiratory infection” refers to bacterial respiratory infections as well as bacterial and viral respiratory co-infections.

As used herein, an “instructional material” includes a publication, a recording, a diagram, or any other medium of expression which can be used to communicate the usefulness of a component of the invention in a kit for detecting biomarkers disclosed herein. The instructional material of the kit of the invention can, for example, be affixed to a container which contains the component of the invention or be shipped together with a container which contains the component. Alternatively, the instructional material can be shipped separately from the container with the intention that the instructional material and the component be used cooperatively by the recipient.

The “level” of one or more biomarkers means the absolute or relative amount or concentration of the biomarker in the sample as determined by measuring mRNA, cDNA or protein, or any portion thereof such as oligonucleotide or peptide.

“Measuring” or “measurement,” or alternatively “detecting” or “detection,” means determining the presence, absence, quantity or amount (which can be an effective amount) of either a given substance within a clinical or subject-derived sample, including the derivation of qualitative or quantitative concentration levels of such substances, or otherwise determining the values or categorization of a subject's clinical parameters.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The terms “respiratory sample” or “respiratory swab sample” as used herein mean any sample from a subject containing RNA or secreted proteins a plurality of which is generated by cells in the respiratory tract. Non-limiting examples include nasal swabs, nasopharyngeal swabs, nasopharyngeal aspirate, oral swab, oropharyngeal swab, pharyngeal (throat) swab, sputum, bronchoalveolar lavage or saliva or transport medium exposed to any of these sample types.

A “reference level” of a biomarker means a level of the biomarker that is indicative of the absence of a particular disease state or phenotype. When the level of a biomarker in a subject is above the reference level of the biomarker it is indicative of the presence of a particular disease state or phenotype. When the level of a biomarker in a subject is within the reference level of the biomarker it is indicative of a lack of a particular disease state or phenotype.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

### Methods

In one aspect, the invention provides a method for detecting and distinguishing among viral-only or a bacterial-associated respiratory infection in a patient, the method includes first analyzing a respiratory sample to determine levels of at least two respiratory virus infection-associated and/or bacterial respiratory infection-associated molecules. Next, the levels of the respiratory virus infection-associated molecules and/or the levels of bacterial respiratory infection-associated molecules are compared with a predetermined reference level for respiratory virus infection-associated molecules and/or a predetermined reference level of the bacterial respiratory infection-associated molecules. Then, after comparing the analyzed levels with the reference levels, the patient is determined to have a respiratory viral infection if the level of the respiratory virus infection-associated molecules is above the respective reference level, or the patient is determined to have a bacterial-associated respiratory infection if the level of the bacterial respiratory infection-associated molecules is above the respective reference level.

Messenger RNAs and proteins which change their level of expression in response to viral or bacterial infection are here called viral respiratory infection-associated or bacterial respiratory infection associated molecules.

### Viral Respiratory Infection-Associated Molecules

A viral respiratory infection-associated molecule may be any molecule the expression of which changes in a patient having a respiratory viral infection relative to a patient that does not have a respiratory viral infection. In some embodiments, the viral associated molecule is an interferon-stimulated gene product. In other embodiments, the viral associated molecule binds to the CXCR3 receptor. In some embodiments, the viral associated molecule is CXCL10.

In some embodiments, the patient is tested for the presence of a common virus prior to screening of the sample for the presence of other disease causing viruses. By way of non-limiting examples, the common virus may be a Rhinovirus, Influenza A and B (IAV, IBV), Parainfluenza 1, 2, and 3 (PIV 1-3), Respiratory syncytial virus A and B (RSV A, B), Human metapneumovirus (hMPV), Adenoviruses (AdV), or Parainfluenza 4 (PIV-4), and SARS-CoV-2.

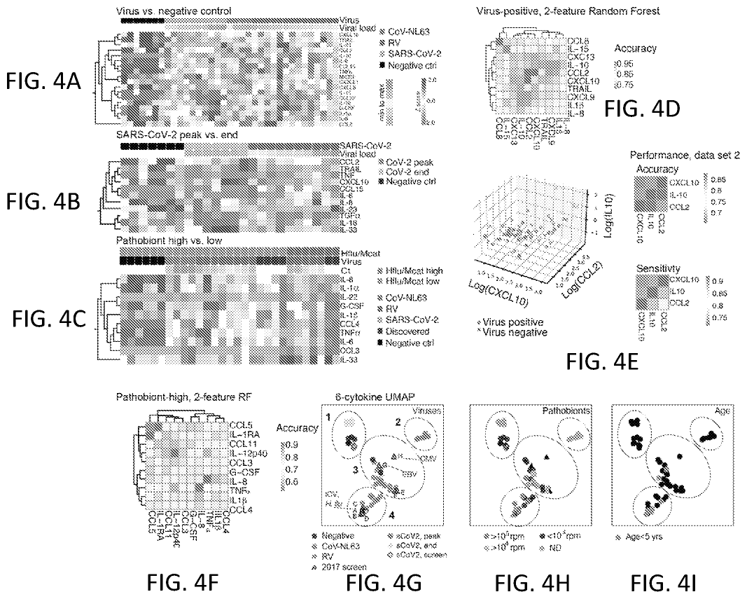

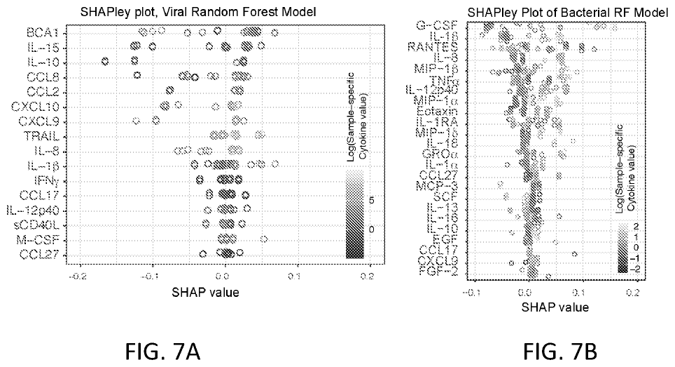

In some embodiments, one or more molecules, such as cytokines, are used as a predictor to detect virus in a sample, such as an NP sample. The evaluation of various respiratory virus infection-associated molecules is illustrated in FIGS. 4A, D, and E, as well as the associated figure legends and the examples presented herein. In some embodiments, the respiratory virus associated molecules comprise any of the molecules listed in FIG. 4A or 7A, or pairs of molecules indicated in FIG. 4D or E. For example, in some embodiments, the respiratory virus infection-associated molecules include CXCL10, TRAIL, IL-23, CCL2, IL-10, IL-6, CCL15, TNFα, M-CSF, CX3CL1, CXCL9, IL-15, CCL22, IL-16, G-CSF, IL-1α, IL-8, CCL8, BCA1, IL-1β, IFNγ, CCL17, IL-12p40, sCD40L, and CCL27. In some embodiments, the respiratory virus infection-associated molecules include CXCL10, TRAIL, IL-23, CCL2, IL-10, IL-6, CCL15, M-CSF, CX3CL1, CXCL9, IL-15, CCL22, IL-16, IL-1α, CCL8, BCA1, IFNγ, CCL17, sCD40L, and CCL27.

Additionally or alternatively, by way of further non-limiting examples, viral infection-associated molecules may be identified by BCA1, IL-15, IL-10, CCL8, CCL2, CXCL10, CXCL9, TRAIL, IL-8, IL-1β, IFNγ, CCL17, IL-12p40, sCD40L, M-CSF, and/or CCL27. In other embodiments, the viral-associated cytokine signature comprises CCL8, IL-15, CXC13, IL-10, CCL2, CXCL10, TRAIL, CXCL9, IL-1β and IL-8. In some embodiments, samples high in CXCL10 that harbor coronaviruses display a common transcriptional signature. In some embodiments, the common, viral-associated cytokine signature comprises IFNγ, LPS, IL1β, NFKβ, IFNα, PolyI:C, TNF, TPA, tretinoin, TGM2, STAT1, IRF7 and IFNα2.

As will be appreciated by those skilled in the art, the at least two respiratory virus infection-associated molecules may include in suitable combination of molecules disclosed herein. In some embodiments, for example, the at least two respiratory virus infection-associated molecules include combinations of at least CXCL10 and CCL2; CXCL10 and IL-10; or CCL2 and IL-10. Although described herein primarily with respect to two molecules, the disclosure is not so limited and may include any other suitable number of respiratory virus infection-associated molecules, such as, but not limited to, 2, 3, 4, 5, 6, 7, 8, 9, 10, 11, 12, 13, 14, 15, 16, 17, 18, 19, 20, 21, 22, 23, 24, or 25 molecules. For example, in some embodiments, the at least two molecules include CXCL10, CCL2, and IL-10. Additionally or alternatively, in some embodiments, the at least two molecules include CXCL10, CCL2, IL-10, CXCL10 and CCL2, CXCL10 and IL-10, or CCL2 and IL-10, in combination with one or more other respiratory virus infection-associated molecules disclosed herein. In some embodiments, the combination of cytokines with the highest level of accuracy in predicting virus infection is determined in a random forest prediction model.

In some embodiments, the expression level of CXCL10 is used as a predictor to detect a respiratory virus other than a common virus in an NP sample. In some embodiments, the viral respiratory infection-associated molecules is CXCL10 which is detected at a higher level in respiratory swabs from patients with a viral infection. In some embodiments, a virus capable of causing human disease is detected in the samples with elevated levels of expression of CXCL10. In some embodiments, the virus capable of causing human disease is an Influenza virus, Epstein-Bar virus, or cytomegalovirus. In some embodiments, a novel virus is detected in samples with elevated CXCL10 levels. In some embodiments, the novel virus is a coronavirus. In some embodiments the coronavirus is SARS-CoV-2. In some embodiments, the virus is a SARS-CoV-2 variant. In some embodiments, a viral infection precedes the elevation of CXCL10 levels in the patient. In other embodiments, elevated CXCL10 levels increase a patient's susceptibility to viral infections.

### Bacterial Respiratory Infection-Associated Molecules

Turning to the bacterial respiratory infection-associated molecules, FIG. 4F, the associated figure legend, and the examples presented herein show a similar evaluation of bacterial respiratory infection-associated molecules. Accordingly, in some embodiments, the bacterial respiratory infection-associated molecules comprise any of the molecules shown in FIG. 4C or 7B, or pairs of molecules indicated in FIG. 4F. For example, in some embodiments, the bacterial respiratory infection-associated molecules include CCL5, IL-1RA, CCL11, IL-12p40, CCL3, G-CSF, IL-8, TNFα, IL-1β, CCL4, IL-1α, IL-22, IL-6, RANTES, MIP-1β, MIP-1α, Eotaxin, GROα, CCL27, MCP-3, SCF, IL-13, IL-16, IL-10, EGF, CCL17, CXCL9, and FGF-2. In some embodiments, the bacterial respiratory infection-associated molecules include CCL5, IL-1RA, CCL11, IL-12p40, CCL3, G-CSF, IL-8, TNFα, IL-1β, CCL4, IL-1α, IL-22, and IL-6. In some embodiments, the bacterial respiratory infection-associated molecules include CCL5, IL-1RA, CCL11, IL-12p40, CCL3, G-CSF, IL-8, TNFα, IL-1β, and CCL4. In some embodiments, the bacterial respiratory infection-associated molecules include TNF, IL-8, and IL-1β.

Similar to the at least two respiratory virus infection-associated molecules, as will be appreciated by those skilled in the art, the at least two bacterial respiratory infection-associated molecules may include in suitable combination of molecules disclosed herein. In some embodiments, for example, the at least two bacterial respiratory infection-associated molecules include combinations of at least TNF and IL-8 or IL-1β and IL-8. Although described herein primarily with respect to two molecules, the disclosure is not so limited and may include any other suitable number of bacterial respiratory infection-associated molecules, such as, but not limited to, 2, 3, 4, 5, 6, 7, 8, 9, 10, 11, 12, 13, 14, 15, 16, 17, 18, 19, 20, 21, 22, 23, 24, or 25 molecules.

Additionally or alternatively, the method may include any combination of at least two respiratory virus infection-associated molecules and at least two bacterial respiratory infection-associated molecules. In some embodiments, for example, the at least two respiratory virus associated molecules comprise CXCL10 and CCL2, and the at least two bacterial respiratory infection-associated molecules include TNF and IL-8 or IL-1β and IL-8. In some embodiments, analyzing a respiratory sample comprises determining levels of at least CXCL10, CCL2, IL-10, IL-8, TNF, and IL-1β. As set forth in Example 8, analysis of these 6 molecules efficiently determines the disease status of patients presenting with symptoms of a respiratory infection.

Without meaning to be limited by theory, the invention is based in part on the discovery that measuring, normalizing and comparing levels of specific markers can determine whether a patient has a viral only respiratory infection or a bacterial respiratory infection or a viral/bacterial coinfection. This is a crucial determination due to the fact that medical professionals must determine if treatment with antibiotics is appropriate. Accordingly, in various embodiments, the patient is determined to have a bacterial-associated respiratory infection and the patient is treated with antibiotics. In various embodiments, the antibiotic may be penicillin, amoxicillin or erythromycin. In various embodiments, the patient is determined to have a respiratory viral infection and the patient is treated with antivirals.

While biomarkers detecting a viral infection have been described using patient blood samples, unexpectedly, markers for host response are easily detectable in respiratory samples, by way of non-limiting example, by using swabs of the upper respiratory tract. In various embodiments, the respiratory swab may be obtained from the nose, nasopharynx, mouth, oropharynx, throat or ears. Release of the material may be aided by stirring, vortexing or any other method known in the art or may simply occur by passive diffusion. Solvent may be of any type known in the art and may comprise various additives to stabilize viruses, bacteria, proteins or other biological materials including but not limited to pH buffers, antibiotics, and/or cryoprotectants such as sucrose. Samples obtained by sampling the upper respiratory tract are much less invasive and are more directly relevant to disease pathogenesis than blood samples in the case of respiratory infection.

Elevated expression of various biomarkers may be detected in these samples at the protein or mRNA level. Accordingly, in some embodiments, expression of biomarkers is determined by measuring the level of mRNA encoding for the molecule. In such embodiments, the respiratory swab sample may be centrifuged to form a pellet of cells and cell debris which is then added to lysis buffer. Total nucleic acid is isolated from the pellet and DNA is digested using, by way of non-limiting example, DNAse I. The RNA is then reverse transcribed into cDNA. The cDNA is then analyzed to determine the level of at least one respiratory infection-associated molecule. In some embodiments the level of the at least one respiratory infection-associated molecule is determined by reverse transcription quantitative polymerase chain reaction (rt-qPCR) although the skilled artisan will appreciate that there are other ways that the level of the at least one respiratory infection-associated molecule may be determined by the analysis of mRNA and these methods are encompassed by the invention in its various embodiments. A skilled person is capable of selecting and practicing an appropriate technique as the measurement of levels of specific mRNAs and proteins in a sample is a familiar operation to a skilled artisan.

Additionally or alternatively, in some embodiments, expression is determined by measuring protein. In such embodiments, the protein level is determined by ELISA, an immunoassay, or by mass spectrometry. In various embodiments the proteins are secreted proteins, in some embodiments the proteins are chemokines.

In some embodiments, the expression level of the measured respiratory infection-associated molecules are normalized to the expression level of a housekeeping gene. The expression level of the housekeeping gene may be measured using the same method as the one or more respiratory infection-associated molecule. In some embodiments, the housekeeping gene is β-actin, HPRT, or GAPDH.

In some embodiments, the method further comprises treating a patient exhibiting symptoms of respiratory infection after determining levels of respiratory infection-associated molecules by measuring either protein or mRNA. In various embodiments, patients exhibiting a level of the biomarker may be treated for respiratory viral infection. In various embodiments, subjects determined to have a viral respiratory infection are treated with antivirals or are treated by monitoring and recommending supportive care, such as rest and fluids.

In other embodiments, host response biomarkers described here could be used to differentiate incidental detection of microorganism(s) in the upper respiratory tract from an active infectious process that the body is fighting. In recent years epidemiological surveys testing for a panel of respiratory viruses have repeatedly found high rates of respiratory virus detection even in the asymptomatic population. Similarly, streptococcal bacteria which cause strep throat illnesses can also be present in the throat without causing symptoms (“carrier state”.) This has raised questions about the usefulness of pathogen-specific tests, in particular sensitive PCR-based tests for pathogen genomes, to determine whether a detected microorganism is causing disease, or if several organisms are detected, which among those is causing disease. The reference level may be set such that it indicates that a respiratory virus or bacterial infection is the cause for the patient's symptoms. Accordingly, in one aspect the invention provides a method of rule in a specific type of active infectious process, in conjunction with cytokine level detection, which pathogen detection alone does not provide.

In various embodiments, the methods described herein may be used in combination with pathogen specific tests in order to, by way of non-limiting example, to determine the identity of the virus that is responsible for the patient's symptoms and to guide treatment. In various embodiments, the pathogen specific tests may detect one or more of influenza A, influenza B, streptococcus, coronavirus and respiratory syncytial virus. In various embodiments, pathogen specific tests for one or more virus may be performed subsequent to an initial screening for the presence of a common virus.

In another aspect, the methods of the invention may be used to detect pre-symptomatic patients suffering from a viral or bacterial associated respiratory infection. Host response based expression changes of viral respiratory infection associated may appear before the disease may be recognized based on the appearance of patient symptoms. Accordingly, in some embodiments, the methods of the invention may be applied to individuals at risk of infection to predict the appearance of symptoms or to people in situations where the appearance of the symptoms of respiratory infection would cause unusually serious problems, by way of non-limiting example, prior to travel or undertaking work that would be compromised by a respiratory infection.

In another aspect, individual biomarkers or biomarker combinations could be used to distinguish between viral-only respiratory infection and infections caused by bacteria or by viral-bacterial co-infection. Bacterial pathogens include H. influenza, M. catarrhalis, and S. pneumoniae. Such biomarkers could be used to guide antibiotic therapy by distinguishing whether infections would require antibiotics for resolution.

As described in further detail below, various respiratory virus infection-associated molecules and bacterial respiratory infection-associated molecules may be analyzed in according to one or more of the embodiments disclosed herein.

### Compositions

In another embodiment, the invention provides biomarker signatures associated with distinct infection-associated immunophenotypes. In another embodiment, measuring NP cytokines increases the efficiency of pathogen discovery diagnosis of infectious diseases. In some embodiments, a biomarker of the interferon response is used to enrich for nasopharyngeal (NP) samples most likely to contain undiagnosed viruses among samples from symptomatic patients testing negative on a standard hospital respiratory virus panel (RVP). In some embodiments, the respiratory virus panel comprises common viruses.

In some embodiments, clinical samples with known infection status are deeply characterized and machine learning is applied to define biomarker signatures of distinct infection-associated immunophenotypes. In some embodiments, the measuring of NP cytokines increases the efficiency of pathogen discovery and improves diagnosis of infectious diseases. In some embodiments, the measuring of NP cytokines increases the scope and efficiency of pathogen detection from clinical samples.

In one embodiment, the composition comprises a solvent. The solvent can be any solvent known to a skilled artisan to be safe for administration to a mammal. In one embodiment, the solvent is an aqueous solvent. Exemplary aqueous solvents include, but are not limited to, tap water, distilled water, deionized water, saline, sterile water, filtered water, and combinations thereof. In some embodiments, the solvent is normal saline. In other embodiments, the solvent is sterile water.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Methods

### Human Experimental Guidelines Approval Statement

Residual nasopharyngeal samples from clinical testing were obtained from the Yale-New Haven Hospital Clinical Virology Laboratory and medical records were reviewed followed by de-identification. data de-identified, and discovered positive SARS-CoV-2 cases were reported to health care providers according to IRB-approved protocol #2000027656 with oversight from the Yale Human Investigations Committee.

### Clinical Samples

We used residual nasopharyngeal (NP) samples remaining after clinical testing for CXCL10 measurements and transcriptome and proteome analysis. Swab-associated viral transport medium was stored at −80° C. following clinical testing and thawed just prior to immunoassay or RNA isolation for RNA-Seq. Clinical information including age, sex, virology and microbiology results, and specific features of clinical course including presenting symptoms, hospital admission and length of stay, was extracted from the electronic medical record and recorded, after which samples were assigned a study code and de-identified.

### Clinical Virology Testing

For testing by the YNHH Clinical Virology Laboratory, NP swabs were placed in viral transport media (BD Universal Viral Transport Medium) immediately upon collection. Samples (200 μL) were subjected to total nucleic acid extraction using the NUCLISENS easyMAG platform (BioMérieux, France). The 10-virus PCR panel was performed as previously described (Morens et al., Cell 182, 1077-1092). CXCL10-high samples from January 2017 were tested for four coronaviruses and PIV4 as described previously (Landry et al., J. Infect. Dis. 217, 897-905, (2018)). The 15-virus PCR panel included updated rhinovirus PCR detection and inclusion of 4 seasonal coronaviruses and parainfluenza virus (Allander et al., PNAS 102, 12891-12896, (2005); Landry et al., J. Infect. Dis. 217, 897-905, (2018); Lu, X. et al., J. Infect. Dis. 216, 1104-1111 (2017); Pierce et al., J Clin Microbiol 50, 364-371 (2012)). YNHH testing for SARS-CoV-2 was done using N1, N2, and RNAse P primer probe sets with an emergency use authorized assay developed by the CDC (CDC 2019-Novel Coronavirus (2019-nCoV) Real-Time RT-PCR Diagnostic Panel (2020)).

### CXCL10 Measurements

For screening, human CXCL10 was measured in duplicate for each sample using the R&D Human CXCL10/IP-10 DuoSet ELISA (Cat No: DY266) and concentrations were calculated from a standard curve on each plate according to manufacturer instructions using GraphPad Prism software. For 2017 samples, ELISA was performed using a 1:5 dilution of the viral transport medium and [CXCL10]>150 pg/ml was considered screen positive. For 2020 samples, ELISA was performed using a 1:1 dilution of the viral transport medium and used a cutoff of 100 pg/ml.

### Library Preparation and RNA Sequencing

At the time of accessioning, the residual viral transport medium from clinical samples was stored at −80° C. Upon thawing, RNA was isolated from 140p1 of transport medium using the Qiagen Viral RNA isolation kit per manufacturer's instructions (Ref: 52904, Qiagen, Germany) and one aliquot was reserved for ELISA. RNA samples were quantified and checked for quality using the Agilent 2100 Bioanalyzer Pico RNA Assay. Library preparation was performed using Kapa Biosystem's KAPA HyperPrep Kit with RiboErase (HMR) in which samples were normalized with a total RNA input of 25 ng. Libraries were amplified using 15 PCR cycles. Libraries were validated using Agilent TapeStation 4200 D1000 assay and quantified using the KAPA Library Quantification Kit for Illumina® Platforms kit. Libraries were diluted to 1.3 nM and pooled at 1.25% each of an Illumina NovaSeq 6000 S4 flowcell using the XP workflow to generate 25M read pairs/sample.

### RNASeq Data Analysis

Low quality reads were trimmed and adaptor contamination was removed using Trim Galore (v0.5.0). Trimmed reads were mapped to the human reference genome (hg38) using HISAT2 (v2.1.0) (Kim et al., Nat Biotechnol 37, 907-915 (2019)). Gene expression levels were quantified using StringTie (v1.3.3b) with gene models (v27) from the GENCODE project (Pertea et al., Nat Biotechnol 33, 290-295 (2015)). Differentially expressed genes (adjusted p value <0.05, fold change cutoff=2) were identified using DESeq2 (v 1.22.1) (Love et al., Genome Biol 15, 550, (2014)). Master DEG list used for transcriptomic analyses was compiled by merging in DEGs determined by DeSeq, based on pairwise comparisons of virus-positive groups (RV, CoV-NL63, SARS-CoV-2, RV, pathobiont low) to virus negative controls and pairwise comparisons of each virus positive group (RV vs. SARS-CoV-2. RV vs CoV-NL63, CoV-NL63 vs. SARS-CoV2) (n=5773 DEG). Pathway analysis and upstream regulators of DEG in pairwise comparisons was visualized using Ingenuity Pathway analysis (version 01-16). Transcription factor motif enrichment analysis was performed using Cytoscape (version 3.8.1) with the iRegulon plug-in (version 1.3) (Janky et al., PLoS Comput Biol 10, e1003731, (2014)).

### Mapping to Viral Reference Genomes

To identify the viral sequences in 2017 RNASeq data, we constructed a hybrid genome consisting of human reference genome (hg38), a curated collection of 16S rRNA sequences from bacteria and archaea in NCBI RefSeq database as of Mar. 30, 2020, and a curated collection of viral genomic sequences in NCBI RefSeq database as of Mar. 30, 2020. Then we indexed this hybrid genome for HISAT2 and aligned the RNA-seq reads, which were processed using Trim Galore, to the hybrid genome using HISAT2 (v2.1.0) (Kim et al., Nature biotechnology 37, 907-915 (2019)). To obtain the reliable numbers of reads that were mapped to the bacterial and viral sequences, we only considered high-quality reads with MAPQ>=60 and excluded reads with 15 or more consecutive polyN bases.

### RT-qPCR for Influenza C

RNA was isolated from 140 μl of cell culture supernatant (in vitro infection) or viral transport medium (clinical samples) as described above, followed by cDNA synthesis using iScript cDNA synthesis kit (BioRad). qPCR was performed using SYBR green iTaq universal (BioRad) per manufacturer's instructions, using the following PCR primers:
ICV S7 gene (F-TCCAAAATGTCCGACAAAACAGT (SEQ ID NO: 1), R-TGCATTTCAGTGCATGTGTCT (SEQ ID NO: 2))
ICV M2 gene (F-GTCTCAGAAAGTGGAAGAACAGC (SEQ ID NO: 3), R-CCAAGGCCAGTAATACCAGCA (SEQ ID NO: 4))
   In Vitro Infections with Sample A.

Primary human nasal epithelial cells (Promocell, Germany) were grown in conventional culture using BEGM media (Lonza, Walkersville, MD, USA), then inoculated with sample A or viral transport medium only. After 7 day incubation, micrographs were taken to record cell appearance and supernatant was stored at −80° C. for RNA isolation and influenza C RT-qPCR.

### SARS-CoV-2 Screening by PCR

For screening of 642 respiratory virus panel (RVP) negative samples from 2020 for SARS-CoV-2 RNA, eluates from easyMag RNA extraction were screened using the US CDC 2019-nCoV N1 primer probe set or the E gene Sarbeco primer probe set, using the following reaction conditions as described previously (IDT, Coralville, Iowa) (Vogels et al., Nat Microbiol 5, 1299-1305 (2020)). We used the Luna Universal Probe One-step RT-qPCR kit (New England Biolabs, Ipswich, MA, USA) with 5 μL of RNA and primer and probe concentrations of 500 nM of forward and reverse primer, and 250 nM of probe. PCR cycler conditions were reverse transcription for 10 minutes at 55° C., initial denaturation for 1 min at 95° C., followed by 40 cycles of 10 seconds at 95° C. and 20 seconds at 55° C. on the Biorad CFX96 qPCR machine (Biorad, Hercules, CA, USA). PCR-positive samples were confirmed by the YNHH clinical laboratories using the full CDC assay as described above.

### SARS-CoV-2 Sequencing and Phylogenetic Analysis

SARS-CoV-2 positive samples were processed for next-generation sequencing as previously described. Total nucleic acid was subjected to cDNA synthesis using SuperScript IV VILO Master Mix (ThermoFischer Scientific, MA, USA) according to the manufacturer's protocol. cDNA was used as input into a highly multiplexed amplicon generation approach for sequencing on the Oxford Nanopore Technologies MinION (ONT, Oxford, UK)(Quick et al., Nat Protoc 12, 1261-1276 (2017)). Samples were barcoded using the Native Barcoding Expansion Pack (ONT, Oxford, UK), multiplexed, and sequenced using R9.4.1 flow cells (ONT, Oxford, UK). The RAMPART software from the ARTIC Network was used to monitor each sequencing run. Runs were stopped when sufficient depth of coverage was achieved to accurately generate a consensus sequence. Following the completion of each sequencing run, raw reads (.fast5 files) were basecalled using Guppy high-accuracy model (v3.5.1, ONT, Oxford, UK). Basecalled FASTQ files were used as input into the ARTIC Networks consensus sequence generation bioinformatic pipeline. Variants to the reference genome were called with nanopolish48. Stretches of the genome that were not covered by 20 or more reads were represented by stretches of NNN's (Loman et al., Nat Methods 12, 733-735, (2015)).

To infer the evolutionary history and origins of the early sampled SARS-CoV-2 genomes, we performed phylogenetic analysis. Sequences were aligned using MAFFT (Katoh & Standley, Mol Biol Evol 30, 772-780, (2013)), and the trees were was inferred using a Maximum Likelihood approach implemented on IQTree (Minh et al., Mol Biol Evol 37, 1530-1534 (2020)), with GTR substitution model and 1000 UFBoot replicates. The trees were plotted using the python package baltic 0.1.6.

### Assessing Microbial Reads Using IDseq

FASTQ files from patient NP sample RNASeq data were uploaded to IDseq for analysis using the metagenomics pipeline. Reads per million (rpm) and genome coverage of the alignments for Moraxella catarrhalis and Haemophilus influenzae were recorded for each sample to assess presence of respiratory pathobionts. Top hits for respiratory viruses were recorded to confirm clinically diagnosed respiratory viral infections or absence of viruses in negative control samples.

### Visualization of RNA-Seq Data

Heatmaps: NP sample transcriptomes were visualized using the Qlucore Omics Explorer (v3.7; Qlucore, Lund, Sweden). A heatmap was generated using the top 2768 DEG differentially expressed genes, determined with the multigroup comparison function (groups: RV, CoV-NL63, SARS-CoV_2, negative control, discovered), p≤0.005, q≤0.05. Heatmap shows unsupervised clustering of 61 samples: discovered in 2017 screen (n=8), SARS-CoV-2 (n=30), CoV-NL63 (n=4), rhinovirus (n=11), and negative controls (n=8). A list of leukocyte subtype-specific gene was generated using scSeq data from Loske et al., 2021 (Loske et al., Nat Biotechnol, (2021)), omitting genes encoding cytokines and transcripts highly expressed in differentiated primary airway epithelia based on scSeq data (Cheemarla, et al., Journal of Experimental Medicine 218 (2021)). Biological processes for each cluster were identified by Gene ontology (GO) using STRING database version 11.5.

UMAPs: The log of RPKM values was calculated and then z-score normalized per gene for all genes identified to be differentially expressed. All log operations were base 10 and performed after a pseudocount was added to all zero values which are calculated per feature as one half the maximum observed for that feature unless specified otherwise. These values were then passed to the UMAP function as implemented in the R UMAP package with the n neighbors parameter set to 5 and default values otherwise to project the data to a 2-dimensional.

### Proteomic Measurements

Cytokines in the discovery sample set were measured using the BioPlex 200 HD71 Human Cytokine Array/Chemokine Array (Eve Technologies, Calgary, AB). NP swab-associated cell free VTM was shipped overnight on dry ice to Eve Technologies for analysis by BioPlex 200 HD71 multiplex immunoassay. Cytokines that were below the lower limit of quantitation were excluded from downstream analyses. For validation of predictive biomarkers of viral infection, CXCL10, CCL2 (MCP1) and IL-10 were measured using a previously-described sample set which had been stored at −80 C, using the Simpleplex assay on the Ella system analyzed by the Simple Plex Explorer software (Protein simple, San Jose, CA) (Aldo et al., Am J Reprod Immunol 75, 678-693(2016)). Results show mean of each sample run in triplicate.

### Visualization of Proteomic Data

NP proteome heatmaps were visualized using the Qlucore Omics Explorer (v3.7; Qlucore, Lund, Sweden). Virus positive samples (RV, SARS-CoV-2, peak or CoV-NL63) vs. virus negative controls, and pathobiont high vs. pathobiont-low samples were compared using two-group comparisons (p value<0.1). SARS-CoV-2 samples were compared using multi-group comparison of three groups: SARS-CoV-2 peak, SARS-CoV-2 end, and negative controls (p-value cutoff <0.01). Within each group, samples are arranged from low to high viral load based on the sample Ct value. For proteome UMAP plots, the log of raw cytokine values was calculated. These values were then passed to the UMAP function as implemented in the R UMAP package with the n neighbors parameter set to 5 and default values otherwise to project the data to a 2-dimensional space.

### Predictive Models

Virus-positive Classification: Cytokine values were log transformed and all samples in which no determination of viral status was made were removed from the dataset. Out of bounds values were re-coded as the maximum observed cytokine value for that cytokine. The subsequent analysis approach matches that used for pathobiont-high classification (below) except the classification task was the determination of whether a sample was derived from a patient identified to have a viral infection. COVID-19, end of infection samples were excluded.

### Validation

Having identified cytokines that allow accurate classification of samples into their appropriate classes, the top 3 cytokines for viral infection classification and the top 6 cytokines for bacterial infection classification were selected for downstream validation in a new cohort. Random forest models using all cytokines for the respective task were trained and performance metrics assessed as previously described.

### Pathobiont High Classification

Cytokine values for were log transformed and all samples in which no determination of bacterial status was made were removed from the dataset. Out of bounds values were re-coded as the maximum observed cytokine value for that cytokine. Data were then partitioned via a 10× cross validation scheme. Random Forest (RF) models (as implemented in the scikit learn Python package) were trained via 10× cross validation to classify samples in which there was a high bacterial load vs. not, providing performance metrics (accuracy, specificity, sensitivity) and the associated variance across the 10 partitions to assess model overfitting. RF models were trained using 10 estimators and the entropy criterion, otherwise default parameters were used (these parameters were used for all RF models used in this manuscript unless otherwise specified).

Feature importances of the trained classifier were assessed using SHAPley analysis via the SHAP package (Lundberg, et al., Nat Mach Intell 2, 56-67, (2020)) implemented in Python (specifically the treeExplainer module) (Lundberg and Lee, in Proceedings of the 31st international conference on neural information processing systems). The top 10 most important features were selected and 10× cross-fold validated RF models were trained for each feature as well as each combination of 2 features generating families of one-feature and two-feature models. Model metrics and their associated variances were obtained. All model performance metrics were then visualized using a heatmap via the pheatmap package in R.

### Example 1: Discovery of Influenza C Virus Isolate in 2017 Using Host Response-Based Screening

To investigate the presence of undiagnosed respiratory viruses in our patient population, NP swab samples testing negative on a laboratory-developed respiratory virus PCR panel at Yale-New Haven Hospital were tested. (RVP; Table 1). (Wu et al., Lancet Microbe 1, e254-e262, (2020)).

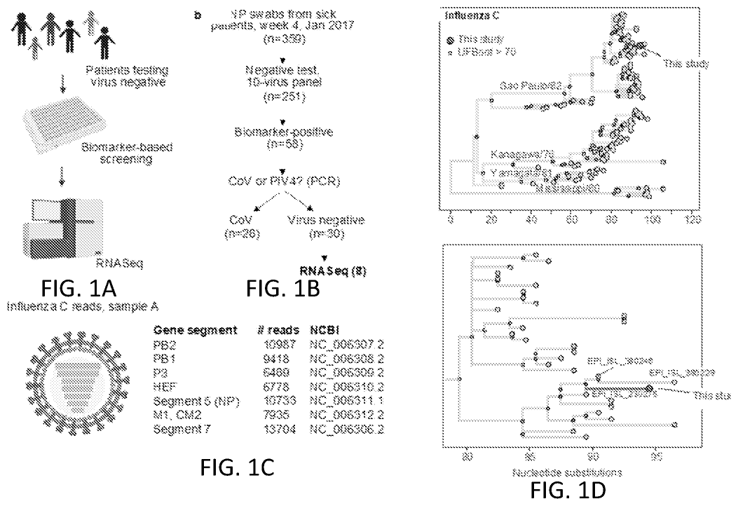

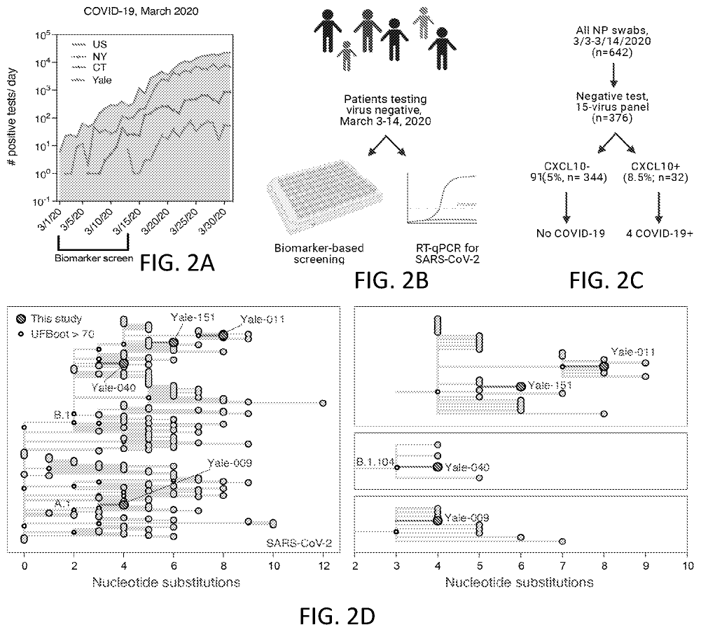

TABLE 1
(related to FIG. 1 and FIG. 2). Respiratory viruses detected by the
Yale-New Haven Hospital PCR panel, 2017 and 2020
Rhinovirus
Influenza A and B (IAV, IBV)
Parainfluenza 1, 2, and 3 (PIV 1-3)
Respiratory syncytial virus A and B (RSV A, B)
Human metapneumovirus (hMPV)
Adenoviruses (AdV)
Parainfluenza 4 (PIV-4)*
Seasonal coronaviruses (CoV-OC43, 229E, NL63, HKU1)*
*tests added in 2019

Due to the large number of RVP-negative samples, samples most likely to contain an undiagnosed viral respiratory infection were first enriched by screening for elevation of the chemokine CXCL10 (FIG. 1A). CXCL10, an interferon stimulated gene, is highly induced in nasal epithelial cells in response to viral RNA and elevated CXCL10 is an excellent predictor of detecting a respiratory virus in an NP sample (Landry & Foxman, The Journal of infectious diseases 217, 897-905, (2018)).

Week 4 of January 2017 was studied, during which 359 nasopharyngeal samples were tested with the RVP. Of those, 251 (70%) were negative for all ten viruses on the RVP in 2017 (FIG. 1B). These samples were from both children and adults, from both inpatient and outpatient settings, with about half presenting with respiratory symptoms and one-third with a history of chronic respiratory illness (Table 2).

TABLE 2
(related to FIG. 1) Patient demographics and clinical presentation
associated with the 251 nasopharyngeal samples testing negative
for respiratory viruses, week 4, January 2017

| | N (%)* | |
|---|---|---|
| Age | | |
| <5 | 9 | (3.6) |
| 6-15 | 2 | (0.8) |
| 16-25 | 13 | (5.2) |
| 26-45 | 28 | (11.2) |
| 46-55 | 35 | (13.9) |
| 56-65 | 56 | (22.3) |
| >65 | 108 | (43.0) |
| Gender | | |
| Male | 98 | (39.0) |
| Female | 153 | (61.0) |
| Race/Ethnicity | | |
| White | 143 | (57.0) |
| Black | 67 | (26.7) |
| Hispanic | 32 | (12.8) |
| Other/Unknown | 9 | (3.6) |
| Patient Status | | |
| Inpatient | 202 | (80.5) |
| Outpatient | 23 | (9.2) |
| ED | 21 | (8.4) |
| Unknown | 5 | (2.0) |
| Presenting symptoms | | |
| Respiratory | 134 | (53.4) |
| Fever | 49 | (19.5) |
| Cardiac | 26 | (10.4) |
| Altered mental state | 22 | (8.8) |
| Fatigue | 8 | (3.6) |
| Other | 95 | (37.8) |
| Comorbidities | | |
| Respiratory | 84 | (33.5) |
| Cardiovascular | 67 | (26.7) |
| Diabetes | 60 | (23.9) |
| Cancer | 51 | (20.3) |
| Liver/kidney disease | 48 | (19.1) |
| Other | 81 | (32.3) |
| Ordering Department | | |
| General medicine | 148 | (59.0) |
| ICU/Surgery | 35 | (13.9) |
| Oncology | 22 | (8.8) |
| ED | 20 | (8.0) |
| Outpatient | 16 | (16.4) |
| Other/Unknown | 10 | (4.0) |

*Percentages may not add up to 100% due to multiple symptoms/comorbidities per single patient

ELISA assay for CXCL10 in the NP swab-associated viral transport media showed that 58 (23%) of the RVP-negative samples had elevated CXCL10. Next, the CXCL10-high samples were tested for common respiratory viral pathogens which were not on the RVP in 2017, including four seasonal coronaviruses (CoV-OC43, NL63, 229E, HKU-1) and parainfluenza virus 4 (PIV4.) About half of the screen-positive samples (28/58) were positive for seasonal coronaviruses (FIG. 1B). Of the remaining screen-positive samples, we focused further analysis on the eight samples with the highest CXCL10 concentrations and therefore the greatest likelihood of containing a respiratory virus (Landry & Foxman, The Journal of infectious diseases 217, 897-905(2018); Table 3).

TABLE 3
Clinical presentation of patients with CXCL10-high NP samples
discovered in January 2017 screen.

| Sample | | | | CXCL10 |
|---|---|---|---|---|
| ID | Age | Sex | Clinical History | (ng/ml) |
| A | 0-5 | M | Acute respiratory illness, cough* | 1.8 |
| B | 0-5 | F | Acute respiratory illness, fever* | 1.4 |
| C | 55-60 | M | Acute respiratory illness, cough, | 0.9 |
| | | | fever* | |
| D | 45-50 | M | Acute respiratory failure, ICU | 0.9 |
| E | 60-65 | F | Fever, COPD, cancer, ICU | 1.1 |
| F | 70-75 | F | ARI, COPD exacerbation | 2.6 |
| G | 20-25 | M | Fever, dyspnea; serology -acute EBV | 0.9 |
| H | 20-25 | F | Fever, rash; serology -acute CMV | 1 |

*outpatient

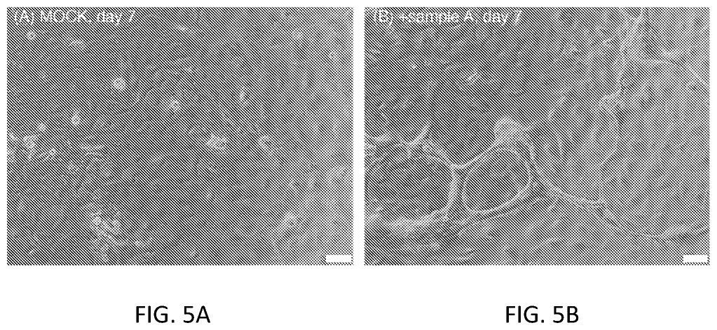

To assess the presence of undiagnosed viruses, ribodepletion RNA sequencing (RNASeq) and read mapping to all viral reference sequences in GenBank was performed. Of the 8 unknowns, one showed over 60,000 reads mapping to the influenza C virus (ICV) reference sequences across all seven genome segments, providing strong support for the presence of ICV (FIG. 1C). Previous reports indicate that ICV primarily causes disease in young children (Thielen et al., Clinical infectious diseases: an official publication of the Infectious Diseases Society of America 66, 1092-1098, (2018). Consistently, this sample came from a 3-year-old outpatient with acute respiratory illness (Patient A, Table 3). ICV was also detected in this sample using RT-qPCR, but was not detected in the other CXCL10-high samples (not shown). Inoculation of primary human nasal epithelial cells with sample A resulted in cell-cell fusion by day 7 post-inoculation (FIG. 5). ICV RNA was also detected in the cell culture supernatant at the day 7. Together, these results support the presence of ICV in the original sample and demonstrate cytopathic effects on primary human nasal epithelial cells.

While no other viruses were identified by RNASeq in the eight NP samples, review of medical records revealed that two of the patients were young adults diagnosed with acute Epstein-Barr virus infection (EBV, Patient G) or acute cytomegalovirus infection (CMV, Patient H) by serology during the patient encounter associated with NP swab collection (Table 3). Thus, acute viral infections were identified in three out of the eight screen-positive samples. In addition to viruses, the presence of RNA from other microbes was examined in these eight samples using the IDSeq platform (Kalantar et al., Giga Science 9, 1-14, (2020)). This analysis revealed abundant RNA from bacterial pathobionts H. influenzae or M. catarrhalis in four of the eight samples, with abundant RNA in two of these samples (samples A, B, C, and F, A and B with >10⁵ reads per million, Table 4). Since these bacteria can cause illness on their own or as co-pathogens with viruses, it is possible that these microbes caused or contributed to the patient symptoms. No pathogens were identified in NP samples D and E, which were from ICU patients with complex clinical courses (Table 3, Table 4).

TABLE 4
(related to FIG. 1). Pathobiont reads associated with 2017
screen-positive NP samples.

| | | reads per | % genome | Depth of |
|---|---|---|---|---|
| Sample | Bacterial pathobiont | million | coverage | coverage |
| A | Haemophilus influenzae | 5.63E+05 | 99.9 | 54.3 |
| | Moraxella catarrhalis | 2.69E+04 | 13.1 | 4.4 |
| B | Moraxella catarrhalis | 3.36E+05 | 64.3 | 36.4 |
| | Haemophilus influenzae | 3.09E+04 | 1.4 | 9.3 |
| C | Haemophilus influenzae | 3.19E+04 | 4.8 | 10.5 |
| D | — | — | — | — |
| E | — | — | — | — |
| F | Haemophilus influenzae | 2.64E+04 | 24.6 | 6.8 |
| G | — | — | — | — |
| H | — | — | — | — |

Finally, to understand the molecular epidemiology of the discovered ICV, phylogenetic analysis based on the hemagglutinin esterase fusion (HEF) gene was performed as in prior studies Thielen et al., Clinical infectious diseases: an official publication of the Infectious Diseases Society of America 66, 1092-1098, (2018; Kalantar, et al., Giga Science 9, 1-14 (2020); Matsuzaki et al., J Clin Microbiol 45, 783-788, (2007)). The analysis placed this isolate within the Sao Paolo serotype (FIG. 1D, upper panel). Comparison to other ICV sequences in the GSAID database showed high similarity to ICVs circulating in Hong Kong and Japan from 2014-2018 (FIG. 1D, lower panel).

### Example 2: Discovery of Undiagnosed SARS-CoV-2 Infections from Early March 2020

Next, it was determined whether a similar screening strategy would be useful for identifying undiagnosed SARS-CoV-2 infections during the early pandemic. The Yale-New Haven Hospital (YNHH) serves southern Connecticut and eastern New York State in the northeastern U.S. The first reported case of COVID-19 in this region occurred on March 2nd (FIG. 2A). Testing began at the hospital on Mar. 13, 2020. We asked whether CXCL10-based screening would detect undiagnosed cases of COVID-19 in early March (FIG. 2B). Of 642 NP samples tested with a 15 respiratory virus panel during this time frame, 376 were negative (59%). A subset of these (32/376; 8.5%) had elevated CXCL10 (FIG. 2C; Table 1). Among the CXCL10-high samples, four were PCR positive for SARS-CoV-2, including a sample from an infant seen as an outpatient (FIG. 2C, Table 5).

TABLE 5
(related to FIG. 2). Clinical presentation of patients with CXCL10 high, respiratory virus panel negative samples from March 2020.

| | Sex | | | | | | | Immuno- | | Admitted | |
|---|---|---|---|---|---|---|---|---|---|---|---|
| Age | (F/M) | Cough/ARI | Fever | Pneumonia | Hypoxemia | COPD | Cancer | suppressed | AKI | to hospital | |
| All (n = 32) | | | | | | | | | | | |
| 65+ (n = 18) | 9/9 | 6 (33%) | 8 (44%) | 6 (33%) | 4 (22%) | 3 (17%) | 2 (11%) | 3 (17%) | 3 (17%) | 16 | (89%) |
| 18-64 (n = 10; | 3/7 | 4 (44%) | 7 (78%) | 2 (22%) | 3 (33%) | 1 (11%) | 1 (11%) | 4 (44%) | 1 (11%) | 7 | (78%) |
| 9 with records) | | | | | | | | | | | |
| 0-18 (n = 5) | 3/2 | 3 (60%) | 3 (60%) | 1 (20%) | 0 | 0 | 0 | 0 | 0 | 2 | (40%) |
| SARS-CoV-2+ (n = 4) | | | | | | | | | | | |

| | | | | | | | | | | |
|---|---|---|---|---|---|---|---|---|---|---|
| 65+ (n = 1) | 1/0 | 1 | 1 | 0 | 0 | 0 | 0 | 1 | 0 | 1 |
| 18-64 (n = 2) | 0/2 | 2 | 2 | 2 | 1 | 1 | 0 | 0 | 0 | 1 |
| 0-18 (n = 1) | 0/1 | 0 | 1 | 0 | 0 | 0 | 0 | 0 | 0 | 0 |

Chart review for the other RVP-negative, CXCL10-high samples showed that one was from a child with acute CMV and EBV infection based on serology which, together with the results of the 2017 screen, suggests that elevated NP CXCL10 may signal acute EBV and/or acute CMV infection in addition to respiratory virus infection. All 344 CXCL10-low samples were negative for SARS-CoV-2 by RT-qPCR.

To ascertain whether these early cases of SARS-CoV-2 in our region were from a single introduction or multiple introductions, whole genome sequencing was performed of using a targeted amplicon strategy described previously (Fauver et al., Cell 181, 990-996, (2020)). Phylogenetic analysis revealed that each isolate was genetically distinct, belonging to different lineages and sub-lineages (FIG. 2D, Tables 6 and 7). Table 6. Summary statistics for MinION sequencing of SARS-CoV-2 positive samples.

TABLE 6
Summary statistics for MinION sequencing of SARS-CoV-2 positive samples.

| | Sample | CT | Reads to | Reads to SARS- | % Reads | Genome | Avg |
|---|---|---|---|---|---|---|---|
| Sample ID | type | Value | Barcode | CoV-2 Genome | Aligned | Coverage | DOC^{b} |
| Yale-009 | NP^{a} | 21 | 123,547 | 123,543 | 100.0 | 97.4 | 200+ |
| Yale-011 | NP | 28 | 159,675 | 156,799 | 98.2 | 96.8 | 200+ |
| Yale-040 | NF | 22 | 345,561 | 342,796 | 99.2 | 98.6 | 200+ |
| Yale-151 | NP | 15 | 243,524 | 239,856 | 98.4 | 99.6 | 200+ |

^{a}NP = nasopharyngeal swab
^{b}DOC = Depth of coverage in reads across SARS-CoV-2 genome

TABLE 7
List of genomes used in the phylogenetic analysis.

| Strain | Clade | Collection Data | Country | Region | Originating lab | Submitting lab | Author |
|---|---|---|---|---|---|---|---|
| Australia/VIC150/2020 | A | 2020 Mar. 17 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC100/2020 | A | 2020 Mar. 16 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC56/2020 | A | 2020 Mar. 12 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC13/2020 | A | 2020 Jan. 31 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC177/2020 | A.1 | 2020 Mar. 18 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC66/2020 | B.4 | 2020 Mar. 13 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC30/2020 | B.4 | 2020 Mar. 10 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC262/2020 | B.1.2 | 2020 Mar. 21 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC294/2020 | B.1.2 | 2020 Mar. 23 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| t | B.1.2 | 2020 Mar. 24 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC237/2020 | B.6 | 2020 Mar. 21 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC276/2020 | B.6 | 2020 Mar. 22 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC188/2020 | B.6 | 2020 Mar. 19 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC231/2020 | B.6 | 2020 Mar. 21 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC251/2020 | B.6 | 2020 Mar. 21 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC176/2020 | B.2 | 2020 Mar. 18 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC280/2020 | B.2 | 2020 Mar. 22 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC305/2020 | B.2 | 2020 Mar. 23 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC268/2020 | B.2 | 2020 Mar. 22 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC10/2020 | B.2 | 2020 Mar. 16 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC179/2020 | B.2 | 2020 Mar. 18 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC194/2020 | B.2 | 2020 Mar. 19 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC169/2020 | B.2 | 2020 Mar. 18 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC203/2020 | B.2.1 | 2020 Mar. 19 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC47/2020 | B.2.1 | 2020 Mar. 12 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC23/2020 | B.2.1 | 2020 Mar. 9 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC21/2020 | B.2.1 | 2020 Mar. 9 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC12/2020 | B.2.1 | 2020 Mar. 16 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC18/2020 | B.2.1 | 2020 Mar. 7 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC149/2020 | B.1 | 2020 Mar. 17 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC219/2020 | B.1 | 2020 Mar. 20 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC90/2020 | B.1 | 2020 Mar. 15 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC278/2020 | B.1.5 | 2020 Mar. 22 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC254/2020 | B.1.5 | 2020 Mar. 21 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC275/2020 | B.1.11 | 2020 Mar. 22 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC174/2020 | B.1 | 2020 Mar. 18 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC63/2020 | B.1 | 2020 Mar. 13 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/VIC201/2020 | B.1 | 2020 Mar. 19 | Australia | Oceania | Victorian Infectious Diseases | Victorian Infectious Diseases Reference Laboratory | Caly et al |
| | | | | | Reference Laboratory (VIDRL) | and Microbiological Diagnostic Unit Public Health | |
| | | | | | | Laboratory, Doherty Institute | |
| Australia/NSW38/2020 | B.6 | 2020 Mar. 10 | Australia | Oceania | Centre for Infectious Diseases | NSW Health Pathology - Institute of Clinical | Chen et al |
| | | | | | and Microbiology Public Health | Pathology and Medical Research; Westmead Hospital; | |
| | | | | | | University of Sydney | |
| Australia/NSW45/2020 | B.6 | 2020 Mar. 13 | Australia | Oceania | Centre for Infectious Diseases | NSW Health Pathology - Institute of Clinical | Eden et al |
| | | | | | and Microbiology Public Health | Pathology and Medical Research; Westmead Hospital; | |
| | | | | | | University of Sydney | |
| Australia/NSW25/2020 | B.6 | 2020 Mar. 5 | Australia | Oceania | Centre for Infectious Diseases | NSW Health Pathology - Institute of Clinical | Gall et al |
| | | | | | and Microbiology Public Health | Pathology and Medical Research; Westmead Hospital; | |
| | | | | | | University of Sydney | |
| Australia/QLD02/2020 | A | 2020 Jan. 30 | Australia | Oceania | Pathology Queensland | Public Health Virology Laboratory | Huang et al |
| Australia/NSW44/2020 | B.6 | 2020 Mar. 10 | Australia | Oceania | Centre for Infectious Diseases | NSW Health Pathology - Institute of Clinical | Rockett et al |
| | | | | | and Microbiology Public Health | Pathology and Medical Research; Westmead Hospital; | |
| | | | | | | University of Sydney | |
| Australia/VIC108/2020 | A | 2020 Mar. 18 | Australia | Oceania | Microbiological Diagnostic Unit | Microbiological Diagnostic Unit Public Health | Seemann et al |
| | | | | | Public Health Laboratory | Laboratory | |
| Australia/VIC290/2020 | B.2 | 2020 Mar. 22 | Australia | Oceania | Microbiological Diagnostic Unit | Microbiological Diagnostic Unit Public Health | Seemann et al |
| | | | | | Public Health Laboratory | Laboratory | |
| Australia/VIC116/2020 | B.2.1 | 2020 Mar. 19 | Australia | Oceania | Microbiological Diagnostic Unit | Microbiological Diagnostic Unit Public Health | Seemann et al |
| | | | | | Public Health Laboratory | Laboratory | |
| Australia/VIC315/2020 | B.1 | 2020 Mar. 24 | Australia | Oceania | Microbiological Diagnostic Unit | Microbiological Diagnostic Unit Public Health | Seemann et al |
| | | | | | Public Health Laboratory | Laboratory | |
| Australia/VIC321/2020 | B.1 | 2020 Mar. 24 | Australia | Oceania | Microbiological Diagnostic Unit | Microbiological Diagnostic Unit Public Health | Seemann et al |
| | | | | | Public Health Laboratory | Laboratory | |
| Belgium/ULG-8634/2020 | B.1 | 2020 Mar. 19 | Belgium | Western | Department of Clinical | GIGA Medical Genomics | Durkin et al |
| | | | | Europe | Microbiology | | |
| Belgium/ULG-9719/2020 | B.1 | 2020 Mar. 22 | Belgium | Western | Department of Clinical | GIGA Medical Genomics | Durkin et al |
| | | | | Europe | Microbiology | | |
| Belgium/VHV-0324118/2020 | B.2 | 2020 Mar. 24 | Belgium | Western | KU Leuven, Clinical and | KU Leuven, Clinical and Epidemiological Virology | Joan Marti- |
| | | | | Europe | Epidemiological Virology | | Carreras et al |
| Belgium/BJ-0324192/2020 | B.1 | 2020 Mar. 24 | Belgium | Western | KU Leuven, Clinical and | KU Leuven, Clinical and Epidemiological Virology | Joan Marti- |
| | | | | Europe | Epidemiological Virology | | Carreras et al |
| Belgium/DA-030691/2020 | B.1 | 2020 Mar. 6 | Belgium | Western | KU Leuven, Clinical and | KU Leuven, Clinical and Epidemiological Virology | Joan Marti- |
| | | | | Europe | Epidemiological Virology | | Carreras et al |
| Belgium/DCS-030796/2020 | B.1 | 2020 Mar. 7 | Belgium | Western | KU Leuven, Clinical and | KU Leuven, Clinical and Epidemiological Virology | Joan Marti- |
| | | | | Europe | Epidemiological Virology | | Carreras et al |
| Belgium/VGA-030672/2020 | B.1 | 2020 Mar. 6 | Belgium | Western | KU Leuven, Clinical and | KU Leuven, Clinical and Epidemiological Virology | Joan Marti- |
| | | | | Europe | Epidemiological Virology | | Carreras et al |
| Belgium/WWM-030665/2020 | B.1 | 2020 Mar. 6 | Belgium | Western | KU Leuven, Clinical and | KU Leuven, Clinical and Epidemiological Virology | Joan Marti- |
| | | | | Europe | Epidemiological Virology | | Carreras et al |
| Belgium/CD-030679/2020 | B.1 | 2020 Mar. 6 | Belgium | Western | KU Leuven, Clinical and | KU Leuven, Clinical and Epidemiological Virology | Joan Marti- |
| | | | | Europe | Epidemiological Virology | | Carreras et al |
| Belgium/MJP-030684/2020 | B.1 | 2020 Mar. 6 | Belgium | Western | KU Leuven, Clinical and | KU Leuven, Clinical and Epidemiological Virology | Joan Marti- |
| | | | | Europe | Epidemiological Virology | | Carreras et al |
| Belgium/PAN-030681/2020 | B.1 | 2020 Mar. 5 | Belgium | Western | KU Leuven, Clinical and | KU Leuven, Clinical and Epidemiological Virology | Joan Marti- |
| | | | | Europe | Epidemiological Virology | | Carreras et al |
| Belgium/RJL-030588/2020 | B.1 | 2020 Mar. 5 | Belgium | Western | KU Leuven, Clinical and | KU Leuven, Clinical and Epidemiological Virology | Joan Marti- |
| | | | | Europe | Epidemiological Virology | | Carreras et al |
| Belgium/LY-030575/2020 | B.1 | 2020 Mar. 5 | Belgium | Western | KU Leuven, Clinical and | KU Leuven, Clinical and Epidemiological Virology | Joan Marti- |
| | | | | Europe | Epidemiological Virology | | Carreras et al |
| Belgium/DRV-0325157/2020 | B.1 | 2020 Mar. 25 | Belgium | Western | KU Leuven, Clinical and | KU Leuven, Clinical and Epidemiological Virology | Joan Marti- |
| | | | | Europe | Epidemiological Virology | | Carreras et al |
| Belgium/BJ-030767/2020 | B.1 | 2020 Mar. 7 | Belgium | Western | KU Leuven, Clinical and | KU Leuven, Clinical and Epidemiological Virology | Joan Marti- |
| | | | | Europe | Epidemiological Virology | | Carreras et al |
| Belgium/HC-030760/2020 | B.1 | 2020 Mar. 7 | Belgium | Western | KU Leuven, Clinical and | KU Leuven, Clinical and Epidemiological Virology | Joan Marti- |
| | | | | Europe | Epidemiological Virology | | Carreras et al |
| Belgium/VHC-030655/2020 | B.1 | 2020 Mar. 6 | Belgium | Western | KU Leuven, Clinical and | KU Leuven, Clinical and Epidemiological Virology | Joan Marti- |
| | | | | Europe | Epidemiological Virology | | Carreras et al |
| Belgium/ULG-3683/2020 | B.2 | 2020 Mar. 7 | Belgium | Western | Department of Clinical | GIGA Medical Genomics | Keith et al |
| | | | | Europe | Microbiology | | |
| Belgium/ULG-6950/2020 | B.1 | 2020 Mar. 15 | Belgium | Western | Department of Clinical | GIGA Medical Genomics | Keith et al |
| | | | | Europe | Microbiology | | |
| Belgium/ULG-6503/2020 | B.1 | 2020 Mar. 13 | Belgium | Western | Department of Clinical | GIGA Medical Genomics | Keith et al |
| | | | | Europe | Microbiology | | |
| Belgium/ULG-6754/2020 | B.1 | 2020 Mar. 14 | Belgium | Western | Department of Clinical | GIGA Medical Genomics | Keith et al |
| | | | | Europe | Microbiology | | |
| Belgium/DB-03023/2020 | B.1 | 2020 Mar. 2 | Belgium | Western | KU Leuven, Clinical and | KU Leuven, Clinical and Epidemiological Virology | Vanmechelen |
| | | | | Europe | Epidemiological Virology | | et al |
| Belgium/SN-03031/2020 | B.1 | 2020 Mar. 3 | Belgium | Western | KU Leuven, Clinical and | KU Leuven, Clinical and Epidemiological Virology | Vanmechelen |
| | | | | Europe | Epidemiological Virology | | et al |
| Belgium/QKJ-03015/2020 | B.1 | 2020 Mar. 1 | Belgium | Western | KU Leuven, Clinical and | KU Leuven, Clinical and Epidemiological Virology | Vanmechelen |
| | | | | Europe | Epidemiological Virology | | et al |
| Belgium/GL-030546/2020 | B.1 | 2020 Mar. 5 | Belgium | Western | KU Leuven, Clinical and | KU Leuven, Clinical and Epidemiological Virology | Vanmechelen |
| | | | | Europe | Epidemiological Virology | | et al |
| Belgium/SS-031047/2020 | B.1 | 2020 Mar. 10 | Belgium | Western | KU Leuven, Clinical and | KU Leuven, Clinical and Epidemiological Virology | Vanmechelen |
| | | | | Europe | Epidemiological Virology | | et al |
| Canada/ON_PHL3919/2020 | B.4 | 2020 Mar. 14 | Canada | North | Public Health Ontario | Public Health Ontario Laboratories | Eshaghi et al |
| | | | | America | Laboratories | | |
| Canada/ON_PHL0976/2020 | B.1 | 2020 Mar. 13 | Canada | North | Public Health Ontario | Public Health Ontario Laboratories | Eshaghi et al |
| | | | | America | Laboratories | | |
| Canada/ON_PHL3692/2020 | B.1 | 2020 Mar. 11 | Canada | North | Public Health Ontario | Public Health Ontario Laboratories | Eshaghi et al |
| | | | | America | Laboratories | | |
| Canada/ON_PHL5757/2020 | B.1.3 | 2020 Mar. 12 | Canada | North | Public Health Ontario | Public Health Ontario Laboratories | Eshaghi et al |
| | | | | America | Laboratories | | |
| Canada/BC_83109/2020 | A.1 | 2020 Mar. 5 | Canada | North | BCCDC Public Health | BCCDC Public Health Laboratory | Eshaghi et al |
| | | | | America | Laboratory | | |
| Canada/BC_4078583/2020 | B.4 | 2020 Mar. 3 | Canada | North | BCCDC Public Health | BCCDC Public Health Laboratory | Eshaghi et al |
| | | | | America | Laboratory | | |
| Canada/BC_9446031/2020 | B.2 | 2020 Mar. 13 | Canada | North | BCCDC Public Health | BCCDC Public Health Laboratory | Eshaghi et al |
| | | | | America | Laboratory | | |
| Canada/NS_13/2020 | B.1 | 2020 Mar. 13 | Canada | North | Queen Elizabeth II Health | National Microbiology Laboratory | Majer et al |
| | | | | America | Science Centre | | |
| Wuhan/WH01/2019 | | 2019 Dec. 26 | China | Eastern Asia | General Hospital of Central | BGI & Institute of Microbiology, Chinese Academy of | Chen et al |
| | | | | | Theater Command of People's | Sciences & Shandong First Medical University & | |
| | | | | | Liberation Army of China | Shandong Academy of Medical Sciences & General | |
| | | | | | | Hospital of Central Theater Command of People's | |
| | | | | | | Liberation Army of China | |
| Guangdong/GZ-S6-P0050/2020 | A | 2020 Feb. 28 | China | Eastern Asia | Guangdong Provincial Institution | Guangdong Provincial Institution of Public Health | Lu et al |
| | | | | | of Public Health, Guangdong | | |
| | | | | | Provinical Center for Disease | | |
| | | | | | Control and Prevention | | |
| Guangdong/2020XN4475-P0042/2020 | A | 2020 Jan. 30 | China | Eastern Asia | Guangdong Provincial Institution | Guangdong Provincial Institution of Public Health | Lu et al |
| | | | | | of Public Health, Guangdong | | |
| | | | | | Provinical Center for Disease | | |
| | | | | | Control and Prevention | | |
| Wuhan/IPBCAMS-WH-01/2019 | | 2019 Dec. 24 | China | Eastern Asia | Institute of Pathogen Biology, | Institute of Pathogen Biology, Chinese Academy of | Ren et al |
| | | | | | Chinese Academy of Medical | Medical Sciences & Peking Union Medical College | |
| | | | | | Sciences & Peking Union | | |
| | | | | | Medical College | | |
| Wuhan/IVDC-HB-04/2020 | | 2020 Jan. 1 | China | Eastern Asia | National Institute for Viral | National Institute for Viral Disease Control and | Tan et al |
| | | | | | Disease Control and Prevention, | Prevention, China CDC | |
| | | | | | China CDC | | |
| Wuhan/IVDC-HB-05/2019 | | 2019 Dec. 30 | China | Eastern Asia | National Institute for Viral | National Institute for Viral Disease Control and | Tan et al |
| | | | | | Disease Control and Prevention, | Prevention, China CDC | |
| | | | | | China CDC | | |
| Sichuan/IVDC-SC-001/2020 | A | 2020 Jan. 15 | China | Eastern Asia | National Institute for Viral | National Institute for Viral Disease Control & | Tan et al |
| | | | | | Disease Control and Prevention, | Prevention, CCDC | |
| | | | | | China CDC | | |
| Shanghai/SH0036/2020 | | 2020 Feb. 4 | China | Eastern Asia | Shanghai Public Health Clinical | National Research Center for Translational Medicine | Wang et al |
| | | | | | Center, Shanghai Medical | (Shanghai), Ruijin Hospital affiliated to Shanghai Jiao | |
| | | | | | College, Fudan University | Tong University School of Medicine & Shanghai | |
| | | | | | | Public Health Clinical Center | |
| Shanghai/SH0117/2020 | A | 2020 Feb. 2 | China | Eastern Asia | Shanghai Public Health Clinical | National Research Center for Translational Medicine | Wang et al |
| | | | | | Center, Shanghai Medical | (Shanghai), Ruijin Hospital affiliated to Shanghai Jiao | |
| | | | | | College, Fudan University | Tong University School of Medicine & Shanghai | |
| | | | | | | Public Health Clinical Center | |
| Shanghai/SH0003/2020 | A | 2020 Jan. 25 | China | Eastern Asia | Shanghai Public Health Clinical | National Research Center for Translational Medicine | Wang et al |
| | | | | | Center, Shanghai Medical | (Shanghai), Ruijin Hospital affiliated to Shanghai Jiao | |
| | | | | | College, Fudan University | Tong University School of Medicine & Shanghai | |
| | | | | | | Public Health Clinical Center | |
| Shanghai/SH0017/2020 | A | 2020 Feb. 1 | China | Eastern Asia | Shanghai Public Health Clinical | National Research Center for Translational Medicine | Wang et al |
| | | | | | Center, Shanghai Medical | (Shanghai), Ruijin Hospital affiliated to Shanghai Jiao | |
| | | | | | College, Fudan University | Tong University School of Medicine & Shanghai | |
| | | | | | | Public Health Clinical Center | |
| Shanghai/SH0128/2020 | A | 2020 Feb. 2 | China | Eastern Asia | Shanghai Public Health Clinical | National Research Center for Translational Medicine | Wang et al |
| | | | | | Center, Shanghai Medical | (Shanghai), Ruijin Hospital affiliated to Shanghai Jiao | |
| | | | | | College, Fudan University | Tong University School of Medicine & Shanghai | |
| | | | | | | Public Health Clinical Center | |
| Shanghai/SH0029/2020 | A | 2020 Feb. 1 | China | Eastern Asia | Shanghai Public Health Clinical | National Research Center for Translational Medicine | Wang et al |
| | | | | | Center, Shanghai Medical | (Shanghai), Ruijin Hospital affiliated to Shanghai Jiao | |
| | | | | | College, Fudan University | Tong University School of Medicine & Shanghai | |
| | | | | | | Public Health Clinical Center | |
| Shanghai/SH0075/2020 | A | 2020 Jan. 30 | China | Eastern Asia | Shanghai Public Health Clinical | National Research Center for Translational Medicine | Wang et al |
| | | | | | Center, Shanghai Medical | (Shanghai), Ruijin Hospital affiliated to Shanghai Jiao | |
| | | | | | College, Fudan University | Tong University School of Medicine & Shanghai | |
| | | | | | | Public Health Clinical Center | |
| Wuhan-Hu-1/2019 | | 2019 Dec. 26 | China | Eastern Asia | | National Institute for Communicable Disease Control | Zhang et al |
| | | | | | | and Prevention (ICDC) Chinese Center for Disease | |
| | | | | | | Control and Prevention (China CDC) | |
| Wuhan/WIV07/2019 | | 2019 Dec. 30 | China | Eastern Asia | Wuhan Jinyintan Hospital | Wuhan Institute of Virology, Chinese Academy of | Zhou et al |
| | | | | | | Sciences | |
| France/HF3138/2020 | B.1 | 2020 Mar. 22 | France | Western | CH Compiegne Laboratoire de | National Reference Center for Viruses of Respiratory | Albert et al |
| | | | | Europe | Biologie | Infections, Institut Pasteur, Paris | |
| France/ARA09428/2020 | B.1 | 2020 Mar. 15 | France | Western | Centre Hospitalier de Macon | CNR Virus des Infections Respiratoires - France SUD | Bal et al |
| | | | | Europe | | | |
| France/ARA11939/2020 | B.1 | 2020 Mar. 21 | France | Western | Institut des Agents Infectieux | CNR Virus des Infections Respiratoires - France SUD | Bal et al |
| | | | | Europe | (IAI), Hospices Civils de Lyon | | |
| France/Lyon_0668/2020 | B.1 | 2020 Mar. 6 | France | Western | Institut des Agents Infectieux | CNR Virus des Infections Respiratoires - France SUD | Bal et al |
| | | | | Europe | (IAI), Hospices Civils de Lyon | | |
| Germany/NRW-24/2020 | B.1 | 2020 Mar. 14 | Germany | Western | Center of Medical Microbiology, | Center of Medical Microbiology, Virology, and | Adams et al |
| | | | | Europe | Virology, and Hospital Hygiene, | Hospital Hygiene, University of Duesseldorf | |
| | | | | | University of Duesseldorf | | |
| Iceland/273/2020 | B.2 | 2020 Mar. 17 | Iceland | Northern | The National University Hospital | deCODE genetics | Gudbjartsson |
| | | | | Europe | of Iceland | | et al |
| Iceland/341/2020 | B.2.2 | 2020 Mar. 15 | Iceland | Northern | deCODE genetics | deCODE genetics | Gudbjartsson |
| | | | | Europe | | | et al |
| Iceland/228/2020 | B.1 | 2020 Mar. 17 | Iceland | Northern | The National University Hospital | deCODE genetics | Gudbjartsson |
| | | | | Europe | of Iceland | | et al |
| Iceland/122/2020 | B.1 | 2020 Mar. 17 | Iceland | Northern | deCODE genetics | deCODE genetics | Gudbjartsson |
| | | | | Europe | | | et al |
| Iceland/186/2020 | B.1 | 2020 Mar. 16 | Iceland | Northern | The National University Hospital | deCODE genetics | Gudbjartsson |
| | | | | Europe | of Iceland | | et al |
| Iceland/208/2020 | B.1.5 | 2020 Mar. 16 | Iceland | Northern | The National University Hospital | deCODE genetics | Gudbjartsson |
| | | | | Europe | of Iceland | | et al |
| Iceland/323/2020 | B.1 | 2020 Mar. 18 | Iceland | Northern | The National University Hospital | deCODE genetics | Gudbjartsson |
| | | | | Europe | of Iceland | | et al |
| Iceland/211/2020 | B.1 | 2020 Mar. 16 | Iceland | Northern | The National University Hospital | deCODE genetics | Gudbjartsson |
| | | | | Europe | of Iceland | | et al |
| Iceland/91/2020 | B.1 | 2020 Mar. 10 | Iceland | Northern | The National University Hospital | deCODE genetics | Gudbjartsson |
| | | | | Europe | of Iceland | | et al |
| Italy/TE4959/2020 | B.1 | 2020 Mar. 14 | Italy | Southern | Presidio ospedaliero “Santo | Istituto Zooprofilattico Sperimentale dell'Abruzzo e | Lorusso et al |
| | | | | Europe | Spirito” | Molise “G. Caporale” | |
| Italy/TE4836/2020 | B.1 | 2020 Mar. 16 | Italy | Southern | Ospedale Civile Giuseppe | Istituto Zooprofilattico Sperimentale dell'Abruzzo e | Lorusso et al |
| | | | | Europe | Mazzini | Molise “G. Caporale” | |
| Italy/UniMI01/2020 | B.1 | 2020 Feb. 24 | Italy | Southern | Laboratory of Infectious | Laboratory of Infectious Diseases, Department of | Zehender et al |
| | | | | Europe | Diseases, Department of | Biomedical and Clinical Sciences L. Sacco, University | |
| | | | | | Biomedical and Clinical Sciences | of Milan | |
| | | | | | L. Sacco, University of Milan | | |
| Luxembourg/LNS0591129/2020 | B.2 | 2020 Mar. 9 | Luxembourg | Western | Laboratoire National de Sante, | Laboratoire National de Sante, Microbiology, | Anke |
| | | | | Europe | Microbiology, Virology | Epidemiology and Microbial Genomics | Wienecke- |
| | | | | | | | Baldacchino et al |
| Luxembourg/LNS0000001/2020 | B.1 | 2020 Feb. 29 | Luxembourg | Western | Laboratoire National de Sante, | Laboratoire National de Sante, Microbiology, | Anke |
| | | | | Europe | Microbiology, Virology | Epidemiology and Microbial Genomics | Wienecke- |
| | | | | | | | Baldacchino et al |
| Luxembourg/LNS3588186/2020 | B.1 | 2020 Mar. 15 | Luxembourg | Western | Laboratoire National de Sante, | Laboratoire National de Sante, Microbiology, | Anke |
| | | | | Europe | Microbiology, Virology | Epidemiology and Microbial Genomics | Wienecke- |
| | | | | | | | Baldacchino et al |
| Luxembourg/LNS2151006/2020 | B.1 | 2020 Mar. 17 | Luxembourg | Western | Laboratoire National de Sante, | Laboratoire National de Sante, Microbiology, | Anke |
| | | | | Europe | Microbiology, Virology | Epidemiology and Microbial Genomics | Wienecke- |
| | | | | | | | Baldacchino et al |
| Luxembourg/LNS7537751/2020 | B.1 | 2020 Mar. 16 | Luxembourg | Western | Laboratoire National de Sante, | Laboratoire National de Sante, Microbiology, | Anke |
| | | | | Europe | Microbiology, Virology | Epidemiology and Microbial Genomics | Wienecke- |
| | | | | | | | Baldacchino et al |
| Netherlands/Utrecht_17/2020 | A.4 | 2020 Mar. 10 | Netherlands | Western | Dutch COVID-19 response team | Erasmus Medical Center | Nieuwenhuijse |
| | | | | Europe | | | et al |
| Netherlands/NoordBrabant_23/2020 | B.2 | 2020 Mar. 5 | Netherlands | Western | Dutch COVID-19 response team | Erasmus Medical Center | Nieuwenhuijse |
| | | | | Europe | | | et al |
| Netherlands/Utrecht_1363564/2020 | B.1 | 2020 Mar. 1 | Netherlands | Western | MHC Utrecht | Erasmus Medical Center | Nieuwenhuijse |
| | | | | Europe | | | et al |
| Netherlands/Utrecht_1363628/2020 | B.1 | 2020 Mar. 1 | Netherlands | Western | MHC Utrecht | Erasmus Medical Center | Nieuwenhuijse |
| | | | | Europe | | | et al |
| Netherlands/Diemen_1363454/2020 | B.1 | 2020 Feb. 28 | Netherlands | Western | RIVM | Erasmus Medical Center | Nieuwenhuijse |
| | | | | Europe | | | et al |
| Netherlands/NA_23/2020 | B.1 | 2020 Mar. 9 | Netherlands | Western | Dutch COVID-19 response team | Erasmus Medical Center | Nieuwenhuijse |
| | | | | Europe | | | et al |
| Netherlands/ZuidHolland_5/2020 | B.1 | 2020 Mar. 4 | Netherlands | Western | Dutch COVID-19 response team | Erasmus Medical Center | Nieuwenhuijse |
| | | | | Europe | | | et al |
| Netherlands/NA_17/2020 | B.1 | 2020 Mar. 9 | Netherlands | Western | Dutch COVID-19 response team | Erasmus Medical Center | Nieuwenhuijse |
| | | | | Europe | | | et al |
| Netherlands/NA_28/2020 | B.1 | 2020 Mar. 12 | Netherlands | Western | Dutch COVID-19 response team | Erasmus Medical Center | Nieuwenhuijse |
| | | | | Europe | | | et al |
| Netherlands/Flevoland_1/2020 | B.1 | 2020 Mar. 9 | Netherlands | Western | Dutch COVID-19 response team | Erasmus Medical Center | Nieuwenhuijse |
| | | | | Europe | | | et al |
| Netherlands/NoordHolland_3/2020 | B.1 | 2020 Mar. 12 | Netherlands | Western | Dutch COVID-19 response team | Erasmus Medical Center | Nieuwenhuijse |
| | | | | Europe | | | et al |
| NewZealand/CoV002/2020 | B.2 | 2020 Mar. 11 | New | Oceania | Dunedin Hospital | University of Otago | M. E |
| | | | | | | | Quinones- |
| | | | Zealand | | | | Mateu et al |
| Portugal/PT0042/2020 | A.2 | 2020 Mar. 17 | Portugal | Southern | CHTMAD | Instituto Nacional de Saude (INSA) | Guiomar et |
| | | | | Europe | | | al et al |
| Spain/VH198152683/2020 | B.1 | 2020 Mar. 24 | Spain | Southern | Hospital Universitari Vall | Hospital Universitari Vall d'Hebron (HUVH) - Vall | Andres et al |
| | | | | Europe | d'Hebron (HUVH) - Vall | d'Hebron Research Institute (VHIR) | |
| | | | | | d'Hebron Research Institute | | |
| | | | | | (VHIR) | | |
| Spain/CastillayLeon201061/2020 | B.1 | 2020 Feb. 26 | Spain | Southern | HOSPITAL GENERAL DE | Instituto de Salud Carlos III | Iglesias- |
| | | | | Europe | SEGOVIA | | Caballero et al |
| Spain/Madrid201105/2020 | B.1 | 2020 Feb. 25 | Spain | Southern | HOSPITAL UNIVERSITARIO | Instituto de Salud Carlos III | Iglesias- |
| | | | | Europe | LA PAZ | | Caballero et al |
| Spain/Valencia42/2020 | B.1.5 | 2020 Mar. 12 | Spain | Southern | Servicio de Microbiologia. | Sequencing and Bioinformatics Service and Molecular | Llucia |
| | | | | Europe | Hospital Clinico Universitario de | Epidemiology Research Group. FISABIO-Public | Martinez- |
| | | | | | Valencia | Health | Priego et al |
| Spain/Valencia37/2020 | A.2 | 2020 Mar. 9 | Spain | Southern | Servicio de Microbiologia. | Sequencing and Bioinformatics Service and Molecular | Maria |
| | | | | Europe | Consorcio Hospital General | Epidemiology Research Group. FISABIO-Public | Alma Bracho et al |
| | | | | | Universitario de Valencia | Health | |
| Spain/Valencia21/2020 | A.2 | 2020 Mar. 10 | Spain | Southern | Servicio de Microbiologia. | Sequencing and Bioinformatics Service and Molecular | Maria |
| | | | | Europe | Consorcio Hospital General | Epidemiology Research Group. FISABIO-Public | Alma |
| | | | | | Universitario de Valencia | Health | Bracho et al |
| Switzerland/GE1422/2020 | B.1 | 2020 Feb. 28 | Switzerland | Western | Hopitaux universitaires de | Hopitaux universitaires de Geneve Laboratoire de | Laubscher |
| | | | | Europe | Geneve Laboratoire de Virologie | Virologie | et al |
| England/20116022902/2020 | B.2 | 2020 Mar. 11 | United | Northern | Respiratory Virus Unit, | Respiratory Virus Unit, Microbiology Services | Galiano et al |
| | | | Kingdom | Europe | Microbiology Services Colindale, | Colindale, Public Health England | |
| | | | | | Public Health England | | |
| England/20124086902/2020 | B.2.2 | 2020 Mar. 18 | United | Northern | Respiratory Virus Unit, | Respiratory Virus Unit, Microbiology Services | Galiano et al |
| | | | Kingdom | Europe | Microbiology Services Colindale, | Colindale, Public Health England | |
| | | | | | Public Health England | | |
| England/20124095702/2020 | B.2 | 2020 Mar. 18 | United | Northern | Respiratory Virus Unit, | Respiratory Virus Unit, Microbiology Services | Galiano et al |
| | | | Kingdom | Europe | Microbiology Services Colindale, | Colindale, Public Health England | |
| | | | | | Public Health England | | |
| England/20134040803/2020 | B.2 | 2020 Mar. 23 | United | Northern | Respiratory Virus Unit, | Respiratory Virus Unit, Microbiology Services | Galiano et al |
| | | | Kingdom | Europe | Microbiology Services Colindale, | Colindale, Public Health England | |
| | | | | | Public Health England | | |
| England/20139051302/2020 | B.2.1 | 2020 Mar. 27 | United | Northern | Respiratory Virus Unit, | Respiratory Virus Unit, Microbiology Services | Galiano et al |
| | | | Kingdom | Europe | Microbiology Services Colindale, | Colindale, Public Health England | |
| | | | | | Public Health England | | |
| England/20100022706/2020 | B.1 | 2020 Feb. 29 | United | Northern | Respiratory Virus Unit, | Respiratory Virus Unit, Microbiology Services | Galiano et al |
| | | | Kingdom | Europe | Microbiology Services Colindale, | Colindale, Public Health England | |
| | | | | | Public Health England | | |
| England/20104002606/2020 | B.1 | 2020 Mar. 3 | United | Northern | Respiratory Virus Unit, | Respiratory Virus Unit, Microbiology Services | Galiano et al |
| | | | Kingdom | Europe | Microbiology Services Colindale, | Colindale, Public Health England | |
| | | | | | Public Health England | | |
| England/20126007002/2020 | B.1.11 | 2020 Mar. 18 | United | Northern | Respiratory Virus Unit, | Respiratory Virus Unit, Microbiology Services | Galiano et al |
| | | | Kingdom | Europe | Microbiology Services Colindale, | Colindale, Public Health England | |
| | | | | | Public Health England | | |
| Scotland/EDB013/2020 | B.1.10 | 2020 Mar. 10 | United | Northern | Virology Department, Royal | Virology Department, Royal Infirmary of Edinburgh, | McHugh et al |
| | | | Kingdom | Europe | Infirmary of Edinburgh, NHS | NHS Lothian | |
| | | | | | Lothian | | |
| Wales/PHWC-24349/2020 | B.2 | 2020 Mar. 18 | United | Northern | Wales Specialist Virology Centre | Public Health Wales Microbiology Cardiff | Moore et al |
| | | | Kingdom | Europe | | | |
| Wales/PHWC-2433A/2020 | B.2 | 2020 Mar. 18 | United | Northern | Wales Specialist Virology Centre | Public Health Wales Microbiology Cardiff | Moore et al |
| | | | Kingdom | Europe | | | |
| Wales/PHWC-24BC9/2020 | B.2.2 | 2020 Mar. 20 | United | Northern | Wales Specialist Virology Centre | Public Health Wales Microbiology Cardiff | Moore et al |
| | | | Kingdom | Europe | | | |
| Wales/PHW27/2020 | B.1 | 2020 Mar. 12 | United | Northern | Wales Specialist Virology Centre | Public Health Wales Microbiology Cardiff | Moore et al |
| | | | Kingdom | Europe | | | |
| Wales/PHWC-24367/2020 | B.1.11 | 2020 Mar. 17 | United | Northern | Wales Specialist Virology Centre | Public Health Wales Microbiology Cardiff | Moore et al |
| | | | Kingdom | Europe | | | |
| England/SHEF-C092F/2020 | B.1 | 2020 Mar. 20 | United | Northern | Virology Department, Sheffield | Department of Infection, Immunity and Cardiovascular | Thushan de |
| | | | Kingdom | Europe | Teaching Hospitals NHS | Disease, The Florey Institute, The Medical School, | Silva et al |
| | | | | | Foundation Trust | University of Sheffield | |
| England/SHEF-C0637/2020 | B.1.5 | 2020 Mar. 26 | United | Northern | Virology Department, Sheffield | Department of Infection, Immunity and Cardiovascular | Thushan de |
| | | | Kingdom | Europe | Teaching Hospitals NHS | Disease, The Florey Institute, The Medical School, | Silva et al |
| | | | | | Foundation Trust | University of Sheffield | |
| USA/WI-07/2020 | A.4 | 2020 Mar. 21 | USA- | North | University of Wisconsin-Madison | University of Wisconsin-Madison AIDS Vaccine | Katarina |
| | | | Midwest | America | AIDS Vaccine Research | Research Laboratories | Braun and |
| | | | | | Laboratories | | Gage |
| | | | | | | | Moreno et al |
| USA/WI-02/2020 | B.1.5 | 2020 Mar. 15 | USA | North | University of Wisconsin-Madison | University of Wisconsin-Madison AIDS Vaccine | Katarina |
| | | | Midwest | America | AIDS Vaccine Research | Research Laboratory | Braun and |
| | | | | | Laboratory | | Gage |
| | | | | | | | Moreno et al |
| USA/WI-22/2020 | A.4 | 2020 Mar. 13 | USA- | North | University of Wisconsin-Madison | University of Wisconsin-Madison AIDS Vaccine | Moreno et al |
| | | | Midwest | America | AIDS Vaccine Research | Research Laboratories | |
| | | | | | Laboratories | | |
| USA/MN59-MDH59/2020 | A.1 | 2020 Mar. 14 | USA- | North | Minnesota Department of Health, | Minnesota Department of Health, Public Health | Plumb et al |
| | | | Midwest | America | Public Health Laboratory | Laboratory | |
| USA/MN34-MDH34/2020 | B.1 | 2020 Mar. 12 | USA- | North | Minnesota Department of Health, | Minnesota Department of Health, Public Health | Plumb et al |
| | | | Midwest | America | Public Health Laboratory | Laboratory | |
| USA/MN5-MDH5/2020 | B.1 | 2020 Mar. 10 | USA- | North | Minnesota Department of Health, | Minnesota Department of Health, Public Health | Plumb et al |
| | | | Midwest | America | Public Health Laboratory | Laboratory | |
| USA/MN43-MDH43/2020 | B.1 | 2020 Mar. 12 | USA- | North | Minnesota Department of Health, | Minnesota Department of Health, Public Health | Plumb et al |
| | | | Midwest | America | Public Health Laboratory | Laboratory | |
| USA/NY-NYUMC18/2020 | B.1 | 2020 Mar. 17 | USA- | North | NYU Langone Health | Department of Pathology and Medicine, New York | Black et al |
| | | | Northeast | America | | University School of Medicine | |
| USA/NY-NYUMC17/2020 | B.1 | 2020 Mar. 17 | USA- | North | NYU Langone Health | Department of Pathology and Medicine, New York | Black et al |
| | | | Northeast | America | | University School of Medicine | |
| USA/NY-NYUMC12/2020 | B.1 | 2020 Mar. 15 | USA- | North | NYU Langone Health | Department of Pathology and Medicine, New York | Black et al |
| | | | Northeast | America | | University School of Medicine | |
| USA/NY-NYUMC5/2020 | B.1.3 | 2020 Mar. 16 | USA- | North | NYU Langone Health | Department of Pathology and Medicine, New York | Black et al |
| | | | Northeast | America | | University School of Medicine | |
| USA/NY-NYUMC20/2020 | B.1 | 2020 Mar. 12 | USA- | North | NYU Langone Health | Department of Pathology and Medicine, New York | Black et al |
| | | | Northeast | America | | University School of Medicine | |
| USA/NY-NYUMC8/2020 | B.1 | 2020 Mar. 17 | USA- | North | NYU Langone Health | Department of Pathology and Medicine, New York | Black et al |
| | | | Northeast | America | | University School of Medicine | |
| USA/NY-NYUMC1/2020 | B.2 | 2020 Mar. 4 | USA- | North | NYU Langone Health | Departments of Pathology and Medicine, New York | Chen et al |
| | | | Northeast | America | | University School of Medicine | |
| USA/NY-NYUMC4/2020 | B.1 | 2020 Mar. 16 | USA- | North | NYU Langone Health | Department of Pathology and Medicine, New York | Chen et al |
| | | | Northeast | America | | University School of Medicine | |
| USA/NY-NYUMC3/2020 | B.1 | 2020 Mar. 16 | USA- | North | NYU Langone Health | Department of Pathology and Medicine, New York | Chen et al |
| | | | Northeast | America | | University School of Medicine | |
| USA/NY-NYUMC2/2020 | B.1 | 2020 Mar. 16 | USA- | North | NYU Langone Health | Department of Pathology and Medicine, New York | Chen et al |
| | | | Northeast | America | | University School of Medicine | |
| USA/NY-NYUMC35/2020 | A | 2020 Mar. 18 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC37/2020 | A | 2020 Mar. 18 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC23/2020 | B.2 | 2020 Mar. 14 | USA- | America | NYU Langone Health | Department of Pathology and Medicine, New York | Maria |
| | | | Northeast | | | University School of Medicine | Aguero- |
| | | | | | | | Rosenfeld et al |
| USA/NY-NYUMC36/2020 | B.2 | 2020 Mar. 18 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC47/2020 | B.2 | 2020 Mar. 19 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC31/2020 | B.2 | 2020 Mar. 17 | USA- | North | NYU Langone Health | Department of Pathology and Medicine, New York | Maria |
| | | | Northeast | America | | University School of Medicine | Aguero- |
| | | | | | | | Rosenfeld et al |
| USA/NY-NYUMC54/2020 | B.1 | 2020 Mar. 19 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC32/2020 | B.1 | 2020 Mar. 17 | USA- | North | NYU Langone Health | Department of Pathology and Medicine, New York | Maria |
| | | | Northeast | America | | University School of Medicine | Aguero- |
| | | | | | | | Rosenfeld et al |
| USA/NY-NYUMC51/2020 | B.1 | 2020 Mar. 18 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC41/2020 | B.1 | 2020 Mar. 18 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC43/2020 | B.1 | 2020 Mar. 18 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC38/2020 | B.1 | 2020 Mar. 18 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC50/2020 | B.1 | 2020 Mar. 18 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC44/2020 | B.1 | 2020 Mar. 18 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC39/2020 | B.1.3 | 2020 Mar. 18 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC49/2020 | B.1.3 | 2020 Mar. 18 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC46/2020 | B.1.3 | 2020 Mar. 18 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC27/2020 | B.1.3 | 2020 Mar. 17 | USA- | North | NYU Langone Health | Department of Pathology and Medicine, New York | Maria |
| | | | Northeast | America | | University School of Medicine | Aguero- |
| | | | | | | | Rosenfeld et al |
| USA/NY-NYUMC25/2020 | B.1.3 | 2020 Mar. 16 | USA- | North | NYU Langone Health | Department of Pathology and Medicine, New York | Maria |
| | | | Northeast | America | | University School of Medicine | Aguero- |
| | | | | | | | Rosenfeld et al |
| USA/NY-NYUMC30/2020 | B.1.3 | 2020 Mar. 17 | USA- | North | NYU Langone Health | Department of Pathology and Medicine, New York | Maria |
| | | | Northeast | America | | University School of Medicine | Aguero- |
| | | | | | | | Rosenfeld et al |
| USA/NY-NYUMC48/2020 | B.1 | 2020 Mar. 18 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY-NYUMC29/2020 | B.1 | 2020 Mar. 17 | USA- | North | NYU Langone Health | Department of Pathology and Medicine, New York | Maria |
| | | | Northeast | America | | University School of Medicine | Aguero- |
| | | | | | | | Rosenfeld et al |
| USA/NY-NYUMC33/2020 | B.1 | 2020 Mar. 17 | USA- | North | NYU Langone Health | Department of Pathology and Medicine, New York | Maria |
| | | | Northeast | America | | University School of Medicine | Aguero- |
| | | | | | | | Rosenfeld et al |
| USA/NY-NYUMC53/2020 | B.1 | 2020 Mar. 19 | USA- | North | NYU Langone Health | Departments of Pathology and Medicine, New York | Maria |
| | | | Northeast | America | | University School of Medicine | Aguero- |
| | | | | | | | Rosenfeld et al |
| USA/NY2-PV08100/2020 | A.2 | 2020 Mar. 4 | USA- | North | MSHS Clinical Microbiology | MSHS Pathogen Surveillance Program | Patel et al |
| | | | Northeast | America | Laboratories | | |
| USA/CT-Yale-009/2020 | A.1 | 2020 Mar. 12 | USA- | This study | Yale Clinical Virology | Grubaugh Lab - Yale School of Public Health | Fauver et al |
| | | | Northeast | | Laboratory | | |
| USA/CT-Yale-040/2020 | B.1.11 | 2020 Mar. 12 | USA- | This study | Yale Clinical Virology | Grubaugh Lab - Yale School of Public Health | Fauver et al |
| | | | Northeast | | Laboratory | | |
| USA/CT-Yale-151/2020 | B.1 | 2020 Mar. 13 | USA- | This study | Ellen Foxman - Lab Medicine | Grubaugh Lab - Yale School of Public Health | Fauver et al |
| | | | Northeast | | | | |
| USA/CT-Yale-011/2020 | B.1.3 | 2020 Mar. 11 | USA- | This study | Ellen Foxman - Lab Medicine | Grubaugh Lab - Yale School of Public Health | Fauver et al |
| | | | Northeast | | | | |
| USA/GA_2741/2020 | B.2 | 2020 Feb. 29 | USA-South | North | GA Department of Public Health | Pathogen Discovery, Respiratory Viruses Branch, | Tao et al |
| | | | | America | Laboratory | Division of Viral Diseases, Centers for Disease | |
| | | | | | | Control and Prevention | |
| USA/TX_2020/2020 | | 2020 Feb. 29 | USA-South | North | Texas Department of State Health | Pathogen Discovery, Respiratory Viruses Branch, | Uehara et al |
| | | | | America | Services Lab Services | Division of Viral Diseases, Centers for Disease | |
| | | | | | | Control and Prevention | |
| USA/WA-S41/2020 | A.1 | 2020 Feb. 28 | USA-West | North | Washington State Department of | Seattle Flu Study | Chu etl al |
| | | | | America | Health | | et al |
| USA/WA-S76/2020 | A.1 | 2020 Mar. 5 | USA-West | North | Washington State Department of | Seattle Flu Study | Chu etl al |
| | | | | America | Health | | et al |
| USA/WA-S68/2020 | A.1 | 2020 Mar. 4 | USA-West | North | Washington State Department of | Seattle Flu Study | Chu etl al |
| | | | | America | Health | | et al |
| USA/WA-UW149/2020 | A.1 | 2020 Mar. 14 | USA-West | North | UW Virology Lab | UW Virology Lab | Roychoudhury |
| | | | | America | | | et al |
| USA/WA-UW98/2020 | A.1 | 2020 Mar. 12 | USA-West | North | UW Virology Lab | UW Virology Lab | Roychoudhury |
| | | | | America | | | et al |
| USA/WA-UW93/2020 | B.6 | 2020 Mar. 11 | USA-West | North | UW Virology Lab | UW Virology Lab | Roychoudhury |
| | | | | America | | | et al |
| USA/WA-NH12/2020 | A.1 | 2020 Mar. 13 | USA-West | North | WA State Department of Health | Pathogen Discovery, Respiratory Viruses Branch, | Tao et al |
| | | | | America | | Division of Viral Diseases, Centers for Disease | |
| | | | | | | Control and Prevention | |
| USA/OR_2656/2020 | | 2020 Feb. 27 | USA-West | North | OR State PHL- | Pathogen Discovery, Respiratory Viruses Branch, | Tao et al |
| | | | | America | Virology/Immunology Section | Division of Viral Diseases, Centers for Disease | |
| | | | | | | Control and Prevention | |
| Vietnam/CM99/2020 | A | 2020 Feb. 11 | Vietnam | Southeastern | National Influenza Center, | National Influenza Center, National Institute of | Le Quynh |
| | | | | Asia | National Institute of Hygiene and | Hygiene and Epidemiology (NIHE) | Mai et al |
| | | | | | Epidemiology (NIHE) | | |

One was part of a cluster of early cases in Connecticut related to early SARS-CoV-2 introductions into Washington state (lineage A.1; Yale-009) (Fauver et al., Cell 181, 990-996, (2020)). The other positive cases belonged to a distinct lineage (B.1) with the spike gene D614G mutation, with two genomes closely related to viruses from New York state (Yale-151 and -011), and one lineage grouping within the sub-lineage B.1.104 (Yale-040), related to viruses from Western Europe. This analysis indicates that SARS-CoV-2 entered our region via multiple independent lines of transmission in early March 2020, consistent with prior studies (Fauver et al., Cell 181, 990-996, (2020)).

### Example 3: Deep Characterization of NP Host Response Using Host and Microbial Metatranscriptomics

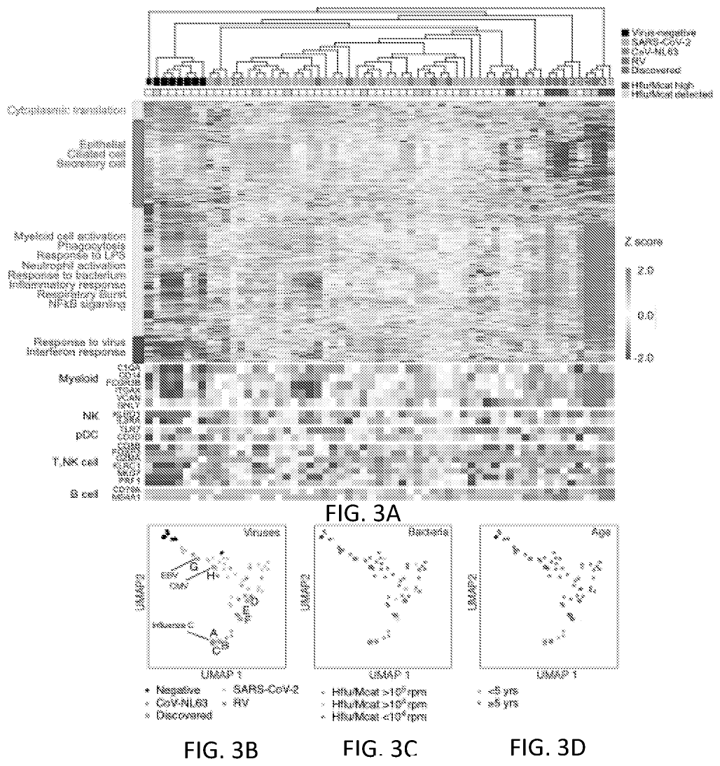

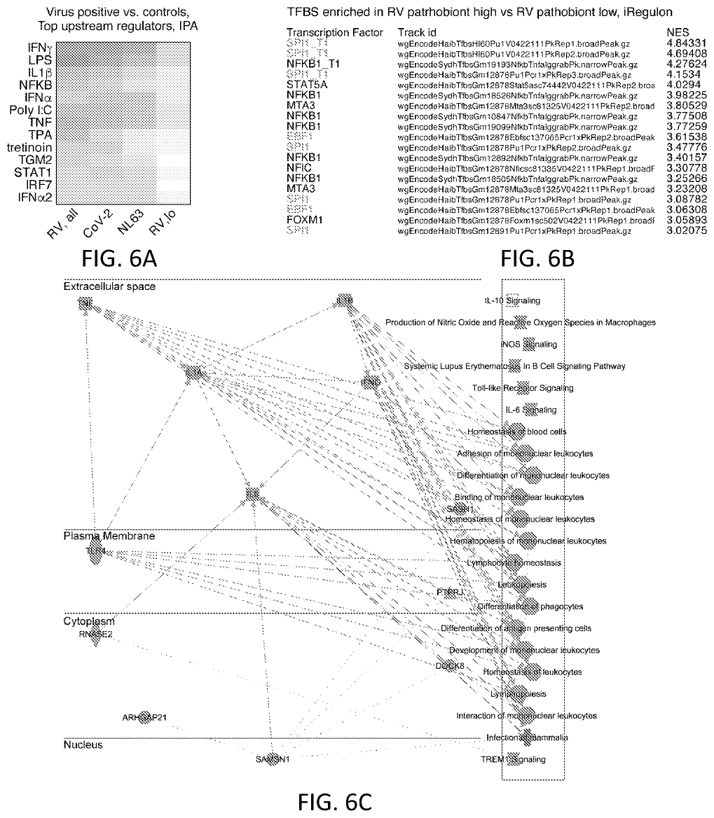

To gain further insight into the NP immunophenotypes associated with infection and/or CXCL10 elevation, the NP transcriptome in known virus-positive and virus-negative samples was evaluated (FIG. 3, FIG. 6). The microbes present were also characterized by mapping the non-host RNA using IDSeq ((Kalantar et al., Giga Science 9, 1-14, (2020); (Table 8). Analysis of genes differentially-expressed between virus-positive samples and negative controls showed that rhinovirus (RV)- and seasonal coronavirus NL-63-(CoV-NL63) positive swabs, together with previously examined SARS-CoV-2 swabs, had a common transcriptional signature characteristic of the antiviral interferon response, with broad induction of interferon stimulated genes (ISGs) (FIG. 6A and prior study (Cheemarla et al., Journal of Experimental Medicine 218 (2021)). Based on enrichment in genes driven by central regulators of innate immunity, such as IRFs, STAT1, and NFKB, RV-positive samples as a group showed more robust induction of innate immune pathways compared to CoV-NL63 or SARS-CoV-2 (FIG. 6A-6C).

TABLE 8
IDSeq analysis of known negative and positive controls

| | | reads | % genome | Depth | | reads | % genome | Depth | | | | |
|---|---|---|---|---|---|---|---|---|---|---|---|---|
| | Bacterial pathobiont | per million | coverage | of coverage | Virus-top hit | per million | coverage | of coverage | Ct value | Age | Sex | Clinical History |
| Negative Controls | | | | | | | | | | | | |
| NC_931 | none | | | | none | | | | | 60-70 | M | |
| NC_932 | none | | | | none | | | | | 60-70 | F | |
| NC_933 | none | | | | none | | | | | 60-70 | F | |
| NC_934 | none | | | | none | | | | | 60-70 | F | |
| NC_936 | Moraxella catarrhalis | 3.62E+05 | 0.5 | 40.3 | none | | | | | 60-70 | F | |
| NC_937 | none | | | | none | | | | | 60-70 | F | |
| NC_939 | Haemophilus parainfluenzae | 3.07E+04 | 4.8 | 3.4 | none | | | | | 70-80 | M | |
| NC_942 | | | | | | | | | | | | |
| Rhinovirus -positive | | | | | | | | | | | | |
| RVRP36 | Moraxella catarrhalis | 5.0E+05 | 60.9 | 24.8 | Rhinovirus C | 11.2 | 45.2 | 0.88 | 29.5 | 0-5 | M | ARI, immunosupressed |
| RVRP31 | Moraxella catarrhalis | 5.1E+05 | 83.9 | 30.3 | Rhinovirus C | 3.6 | 13.3 | 0.26 | 30.3 | 40-50 | F | ARI, immunosuppressed |
| RVRP29 | Haemophilus influenzae | 5.1E+05 | 27.3 | 4.7 | Rhinovirus C | 61.1 | 99.5 | 4.9 | 26.1 | 0-5 | F | ARI, outpatient |
| | Moraxella catarrhalis | 2.0E+05 | 63.8 | 11.7 | | | | | | | | |
| RVRP24 | Haemophilus influenzae | 7.4E+05 | 95.6 | 59.3 | Rhinovirus A | 299.9 | 99.8 | 54.2 | 24 | 0-5 | F | ARI, outpatient |
| | | | | | HRSV/B/Buenos | 911.2 | 100 | 78.9 | | | | |
| | | | | | Aires 2016 | | | | | | | |
| | | | | | Human | 118.4 | 88 | 5.3 | | | | |
| | | | | | coronavirus NL63 | | | | | | | |
| RVRP20 | Haemophilus influenzae | 1.0E+05 | 1.7 | 12.5 | Rhinovirus A | 2541.7 | 99.4 | 244.3 | 22.1 | 90-100 | F | Pneumonia |
| RVRP27 | none | | | | Rhinovirus A | 1131 | 99.3 | 146.5 | 24.5 | 60-70 | F | ARI |
| RVRC312 | none | | | | Rhinovirus C | 210713.8 | 97.1 | 6702.4 | 19 | 20-30 | F | Exacerbation of |
| | | | | | | | | | | | | chronic lung disease |
| RVRP22 | none | | | | Rhinovirus C | 3290.1 | 100 | 612.1 | 22.9 | 70-80 | M | ARI |
| RVRC318 | none | | | | Rhinovirus A | 1766 | 99.7 | 1064 | 22.4 | 20-30 | F | Asthma exacerbation |
| RV922 | none | | | | Rhinovirus C | 2358.9 | 99.8 | 619 | 26.2 | 29 | F | ARI |
| RV927 | none | | | | Rhinovirus C | 67.2 | 99.6 | 32.6 | 27.8 | 44 | F | ARI |
| Seasonal CoV-NL63 | | | | | | | | | | | | |
| CoVLRRC 260 | none | | | | Human | 502363.8 | 100 | 2338.7 | 13.4 | 50-60 | M | Fever, cancer |
| | | | | | coronavirus NL63 | | | | | | | |
| CoVLRRC 261 | none | | | | Human | 107740.8 | 100 | 6009.2 | 13.8 | 50-60 | M | Cough, immunosppressed |
| | | | | | coronavirus NL63 | | | | | | | |
| NC928 | none | | | | Human | 252.4 | 35.1 | 7.8 | 19.6 | 11 | M | ARI, dehydration |
| | | | | | coronavirus NL63 | | | | | | | |
| NC929 | none | | | | Human | 2743.3 | 100 | 398.3 | 14 | 4 | M | ARI, cancer |
| | | | | | coronavirus NL63 | | | | | | | |

| | | reads | % genome | Depth | | reads | % genome | Depth | | | | |
|---|---|---|---|---|---|---|---|---|---|---|---|---|
| COVID-19 | Bacterial pathobiont | per million | coverage | of coverage | Virus-top hit | per million | coverage | of coverage | Ct value | Age | Sex | Inpatient vs. Outpatient |
| NC830 | Moraxella catarrhalis | 1.8E+05 | 0.1 | 10.3 | Severe acute | 51372.5 | 95.4 | 2600.8 | 17.5 | 40-50 | F | Inpatient |
| | | | | | respiratory | | | | | | | |
| | | | | | syndrome-related | | | | | | | |
| | | | | | coronavirus | | | | | | | |
| NC853 | Haemophilus parainfluenzae * | 1.3E+05 | 69 | 10.6 | Severe acute | 303.1 | 85.4 | 11.7 | 20.5 | 20-30 | F | Outpatient |
| | | | | | respiratory | | | | | | | |
| | | | | | syndrome-related | | | | | | | |
| | | | | | coronavirus | | | | | | | |
| NC862 | Haemophilus parainfluenzae * | 9.5E+04 | 49.3 | 9.9 | Severe acute | 9.3 | 5.9 | 0.14 | 26.8 | 20-30 | M | Inpatient |
| | | | | | respiratory | | | | | | | |
| | | | | | syndrome-related | | | | | | | |
| | | | | | coronavirus | | | | | | | |
| NC871 | Haemophilus parainfluenzae * | 1.3E+05 | 50.1 | 5.6 | Severe acute | 73324.0 | 99.8 | 1274.3 | 13 | 50-60 | M | Outpatient |
| | | | | | respiratory | | | | | | | |
| | | | | | syndrome-related | | | | | | | |
| | | | | | coronavirus | | | | | | | |
| NC872 | Haemophilus parainfluenzae * | 1.3E+04 | 67.5 | 3.9 | Severe acute | 6327.9 | 99.9 | 286.9 | | | | |
| | | | | | respiratory | | | | | | | |
| | | | | | syndrome-related | | | | 17.7 | 40-50 | M | Outpatient |
| | | | | | coronavirus | | | | | | | |
| NC876 | Haemophilus parainfluenzae * | 1.3E+05 | 12 | 4.5 | Severe acute | 23881.1 | 99.8 | 520.8 | 15.3 | 20-30 | M | Outpatient |
| | | | | | respiratory | | | | | | | |
| | | | | | syndrome-related | | | | | | | |
| | | | | | coronavirus | | | | | | | |
| COVID19RC282 | Moraxella catarrhalis | 1.9E+05 | 0.5 | 14.7 | Severe acute | 10537.2 | 100 | 785 | 18.3 | 70-80 | M | Outpatient |
| | | | | | respiratory | | | | | | | |
| | | | | | syndrome-related | | | | | | | |
| | | | | | coronavirus | | | | | | | |
| COVID19RC250 | none | | | | Severe acute | 92039.3 | 99.4 | 5595 | NA | 60-70 | M | Inpatient |
| | | | | | respiratory | | | | | | | |
| | | | | | syndrome-related | | | | | | | |
| | | | | | coronavirus | | | | | | | |
| COVID19RC251 | none | | | | Severe acute | 194981.7 | 76.7 | 4277.8 | 12.3 | 80-90 | F | Inpatient |
| | | | | | respiratory | | | | | | | |
| | | | | | syndrome-related | | | | | | | |
| | | | | | coronavirus | | | | | | | |
| COVID19RC253 | none | | | | Severe acute | 340.8 | 81.6 | 8.1 | 24.7 | 60-70 | M | Inpatient |
| | | | | | respiratory | | | | | | | |
| | | | | | syndrome-related | | | | | | | |
| | | | | | coronavirus | | | | | | | |
| | none | | | | Rhinovirus C | 17.4 | 99.8 | 24.5 | | | | |
| COVID19RC256 | none | | | | Severe acute | 6890.7 | 99.8 | 814 | 20.7 | 90-100 | F | Inpatient |
| | | | | | respiratory | | | | | | | |
| | | | | | syndrome-related | | | | | | | |
| | | | | | coronavirus | | | | | | | |
| COVID19RC269 | none | | | | Severe acute | 220686.9 | 99.8 | 5578.8 | 17.2 | 70-80 | M | Inpatient |
| | | | | | respiratory | | | | | | | |
| | | | | | syndrome-related | | | | | | | |
| | | | | | coronavirus | | | | | | | |
| COVID19RC272 | none | | | | Severe acute | 2.5 | NA | NA | 31.6 | 30-40 | M | Inpatient |
| | | | | | respiratory | | | | | | | |
| | | | | | syndrome-related | | | | | | | |
| | | | | | coronavirus | | | | | | | |
| COVID19RC273 | none | | | | Severe acute | 9426.1 | 99.8 | 477.5 | 18.1 | 80-90 | M | Inpatient |
| | | | | | respiratory | | | | | | | |
| | | | | | syndrome-related | | | | | | | |
| | | | | | coronavirus | | | | | | | |
| COVID19RC275 | none | | | | Severe acute | 11.9 | 3.5 | 0.11 | 32 | 50-60 | F | Inpatient |
| | | | | | respiratory | | | | | | | |
| | | | | | syndrome-related | | | | | | | |
| | | | | | coronavirus | | | | | | | |
| COVID19RC276 | none | | | | Severe acute | 6572.3 | 99.9 | 531.2 | 20.8 | 70-80 | F | Inpatient |
| | | | | | respiratory | | | | | | | |
| | | | | | syndrome-related | | | | | | | |
| | | | | | coronavirus | | | | | | | |
| COVID19RC278 | Moraxella catarrhalis | 3.2E+04 | 2.4 | 4.7 | Severe acute | 14547.2 | 99.8 | 658.7 | 17.9 | 90-100 | F | Inpatient |
| | | | | | respiratory | | | | | | | |
| | | | | | syndrome-related | | | | | | | |
| | | | | | coronavirus | | | | | | | |
| COVID19RC281 | none | | | | Severe acute | 17409.6 | 99.9 | 531.3 | 20.8 | 30-40 | M | Inpatient |
| | | | | | respiratory | | | | | | | |
| | | | | | syndrome-related | | | | | | | |
| | | | | | coronavirus | | | | | | | |
| COVID19RC283 | Moraxella catarrhalis | 4.5E+04 | 1.3 | 10.7 | Severe acute | 2083.5 | 99.8 | 77.4 | 23 | 60-70 | M | Inpatient |
| | | | | | respiratory | | | | | | | |
| | | | | | syndrome-related | | | | | | | |
| | | | | | coronavirus | | | | | | | |
| NC644 | none | | | | Severe acute | 160815.0 | 70.2 | 282.8 | | 60-70 | M | Inpatient |
| | | | | | respiratory | | | | | | | |
| | | | | | syndrome-related | | | | | | | |
| | | | | | coronavirus | | | | | | | |
| NC873 | Haemophilus parainfluenzae * | 8.1E+04 | 17.6 | 11 | Severe acute | 39837.7 | 99.9 | 1239 | 16.1 | 30-40 | M | Outpatient |
| | | | | | respiratory | | | | | | | |
| | | | | | syndrome-related | | | | | | | |
| | | | | | coronavirus | | | | | | | |
| NC848 | none | | | | Severe acute | 6847.7 | 99.8 | 205.3 | 23.2 | 30-40 | M | Inpatient |
| | | | | | respiratory | | | | | | | |
| | | | | | syndrome-related | | | | | | | |
| | | | | | coronavirus | | | | | | | |
| NC874 | Haemophilus parainfluenzae * | 2.3E+04 | 4.4 | 3.7 | Severe acute | 5389.2 | 99 | 170.3 | 17.9 | 60-70 | F | Outpatient |
| | | | | | respiratory | | | | | | | |
| | | | | | syndrome-related | | | | | | | |
| | | | | | coronavirus | | | | | | | |
| NC855 | Haemophilus parainfluenzae * | 1.7E+04 | 1 | 4.5 | Severe acute | 12608.3 | 99.8 | 673.2 | 22.7 | 60-70 | M | Inpatient |
| | | | | | respiratory | | | | | | | |
| | | | | | syndrome-related | | | | | | | |
| | | | | | coronavirus | | | | | | | |
| NC817 | none | | | | Severe acute | 164033.8 | 98.3 | 5246.1 | 14.5 | 60-70 | M | Inpatient |
| | | | | | respiratory | | | | | | | |
| | | | | | syndrome-related | | | | | | | |
| | | | | | coronavirus | | | | | | | |
| NC833 | none | | | | Severe acute | 19613.1 | 99.8 | 450.2 | 19.3 | 60-70 | M | Inpatient |
| | | | | | respiratory | | | | | | | |
| | | | | | syndrome-related | | | | | | | |
| | | | | | coronavirus | | | | | | | |
| NC865 | none | | | | Severe acute | 1873.0 | 99.7 | 319.8 | 19.7 | 70-80 | M | Inpatient |
| | | | | | respiratory | | | | | | | |
| | | | | | syndrome-related | | | | | | | |
| | | | | | coronavirus | | | | | | | |
| NC816 | none | | | | Severe acute | 61489.0 | 97.7 | 2762.2 | 16.2 | 70-80 | M | Inpatient |
| | | | | | respiratory | | | | | | | |
| | | | | | syndrome-related | | | | | | | |
| | | | | | coronavirus | | | | | | | |
| NC815 | Moraxella catarrhalis | 7.7E+04 | 7.6 | 6.2 | Severe acute | 69987.1 | 96 | 3983.2 | 14.6 | 70-80 | F | Inpatient |
| | | | | | respiratory | | | | | | | |
| | | | | | syndrome-related | | | | | | | |
| | | | | | coronavirus | | | | | | | |
| NC858 | Moraxella catarrhalis | 1.5E+04 | 0.2 | 12.1 | Severe acute | 12.6 | 38.1 | 1.7 | 28.7 | 90-100 | F | Inpatient |
| | | | | | respiratory | | | | | | | |
| | | | | | syndrome-related | | | | | | | |
| | | | | | coronavirus | | | | | | | |

Notably, analysis of bacterial RNA reads in the same samples showed that a subset of rhinovirus-positive samples had high read counts, defined as >10⁵ reads per million (rpm) and >1% bacterial genome coverage, for upper respiratory pathobionts H. influenzae or M. catarrhalis. The RV positive, pathobiont high samples showed robust innate immune response, whereas the RV positive, pathobiont low samples had relatively lower expression of innate immune response transcripts, compared to other virus positive sample groups (FIG. 7A). Direct comparison of RV positive, pathobiont high and RV positive, pathobiont low samples using iRegulon showed enrichment for targets of leukocyte-specific transcription factors in RV positive, pathobiont high samples, indicating an increase in leukocytes in the nasopharynx (FIG. 7B). Ingenuity pathway analysis identified pro-inflammatory cytokines TNF, IL1α, IL1β, and IL6 as major drivers of the genes enriched in RV positive, pathobiont high compared to RV positive, pathobiont low samples (FIG. 7C). Differentially enriched pathways included processes associated with myeloid cell defense against bacteria, such as phagocytosis and production of reactive oxygen species. Taken together, these results suggest that overgrowth of these pathobionts is associated with leukocyte infiltration of the nasopharynx and a heightened innate immune response.

To visualize gene expression patterns, a merged list was created of DEGs derived from pairwise comparisons between RV, CoV-NL63, or SARS-CoV-2 positive samples and virus-negative controls and performed unsupervised clustering of all samples using this gene list (FIG. 3A). Gene expression patterns segregated NP samples into three major groups: (i) virus-negative controls enriched for airway epithelial genes without induction of innate immune responses (left of heatmap; FIG. 3A), (ii) virus-positive samples enriched for airway epithelial genes and an interferon response signature (center of heatmap; FIG. 3A), and (iii) samples with heightened innate immunity, with enrichment for IRF and NFKB targets and leukocyte-specific transcription factors (right of heatmap; FIG. 3A). The latter cluster included the RV positive, pathobiont high samples. To estimate leukocyte recruitment, we used a list of leukocyte-specific transcripts from a prior scSeq study of nasopharyngeal swabs (Loske et al., Nat Biotechnol, in press (2021)), removing cytokine genes and genes highly expressed in airway epithelial cells (Cheemarla et al., Journal of Experimental Medicine 218 (2021)). Samples with viral infection showed enrichment of leukocyte-specific transcripts compared to control subjects, and samples with heightened innate immunity signature (right of heatmap) showed a further increase in leukocyte-specific transcripts including a strong myeloid cell signature, consistent with activation of antibacterial defenses in pathobiont high compared to RV, pathobiont low samples (FIG. 3A, lower panel, FIG. 7, and Table 9)(Cheemarla et al., Journal of Experimental Medicine 218 (2021); Loske et al., Nat Biotechnol, in press (2021)).

TABLE 9
Leukocyte-specific transcripts that identify immune cell subsets in
NP samples (adapted and modified from Loske J et al., 2021)

| T/NK | Neutrophils/ | | | |
|---|---|---|---|---|
| cells | Monocytes/DCs | NK cells | Neutrophils | B cells |
| CD8B | IL3RA | NKG7 | CPA3 | MS4A1 |
| CD3D | TLR7 | GNLY | FCGR3B | CD79A |
| CD27 | CLEC4C | KLRD1 | ITGAX | CD19 |
| GZMK | FCER1A | | | |
| CTLA4 | C1QA | | | |
| FOXP3 | VCAN | | | |
| GZMA | CD14 | | | |
| PRF1 | | | | |
| KLRC1 | | | | |

### Example 4: 2017 Screen-Positive Samples Cluster into Distinct Infection-Related Immunophenotypes

Three of the CXCL10-high samples from the 2017 screen (E, H, and G, purple squares; FIG. 3A) co-clustered with virus-positive samples, showing an epithelial cell signature and enrichment for transcripts associated with the interferon response. Among these, the signatures from CMV- and EBV-positive samples (G and H) were closest to the negative controls. Five samples showed the heightened innate immunity signature (A, B, C, D, and F). Four of these showed significant reads from bacterial pathobionts H. influenzae and/or M. catarrhalis. Bacterial reads were highest in sample A, the ICV positive sample, (H. influenzae>10⁵ rpm, with almost complete coverage of the bacterial genome) and Sample B (M. catarrhalis>10⁵ rpm (Table 3 and Table 4). Samples C and F also showed the heightened innate immunity pattern, albeit to a lesser extent, and reads from H. influenzae (>10⁴ rpm). Samples A, B, and C were from outpatients with acute respiratory infection (ARI) symptoms (Table 3), and sample F was from a subject with chronic obstructive pulmonary disease (COPD) exacerbation. RV positive, pathobiont high samples co-clustered with these samples and were also from outpatients with ARI symptoms (Table 8; Appendix 2). Sample D, which was from an adult intensive care unit (ICU) patient with acute respiratory distress syndrome, also showed the heightened innate immunity pattern, but minimal pathobiont reads (Table 3, Table 4). These results indicate that various clinical scenarios can result in the heightened NP innate immunity pattern, but that for outpatients with ARI, this pattern is associated with increased NP bacterial load of known airway pathobionts.

### Example 5: Heightened Innate Immunity and Pathobiont Enrichment Correlates with Young Age

Host responses were also visualized using Uniform Manifold Approximation and Projection (UMAP) dimensionality reduced transcriptome patterns (FIG. 3B-3D). Consistent with the heatmap, distinct clusters were observed for no infection, viral infection, and pathobiont-high samples. We also examined patient age, since several recent studies have reported a heightened NP innate immunity in children compared to adults with COVID-19 infection (Loske et al., Nat Biotechnol, in press (2021); Pierce et al., JCI Insight 6, 10; 6(9):e148694 (2021)). By overlaying age on the UMAP plot, it was observed that young age (<5 yrs) correlated with both the pathobiont-high status and heightened innate immune response (FIG. 3d). Notably, pathobiont-high NP samples from adults also fell within the cluster associated with heightened innate immunity, and a pathobiont-low sample from a patient <5 yrs was not in this cluster, suggesting that while the heightened innate immune phenotype is an associated with young age, bacterial pathobionts may drive this pattern independently of age.

### Example 6: Proteomic Signatures Recapitulate Immunophenotypes Seen with Transcriptomics

Since protein biomarkers are more practical than transcriptomic signatures for clinical testing, we next sought to define NP cytokine signatures of infection-relevant immunophenotypes. To this end, we performed 71-plex cytokine assays on samples identified in the 2017 and 2020 screens and control samples, including some samples used for transcriptome analysis, and some additional samples including previously described paired NP samples from the peak and end of COVID-19 infection (Table 10; Cheemarla et al., Journal of Experimental Medicine 218 (2021).

TABLE 10
Samples used for proteomics analysis, FIG. 4.

| | | | | | | | Ct value | | |
|---|---|---|---|---|---|---|---|---|---|
| | | Virus | | | | | (low = high | Viral | |
| Sample # | Category | positive? | Hflu/Mcat? | Age | | Gender | viral load) | load | Clincial symptoms |
| 1 | Control, adult | NO | NO | 60s | | M | — | — | NO - covid screen neg |
| 2 | Control, adult | NO | NO | 60s | | F | — | — | NO - covid screen neg |
| 3 | Control, adult | NO | NO | 60s | | F | — | — | NO - covid screen neg |
| 4 | Control, adult | NO | NO | 70s | | M | — | — | NO - covid screen neg |
| 5 | Control, adult | NO | Detected | 60s | | F | — | — | NO - covid screen neg |
| 6 | Control, adult | NO | NO | 60s | | F | — | — | NO - covid screen neg |
| 7 | Control, adult | NO | NO | 60s | | F | — | — | NO - covid screen neg |
| 8 | CoV-NL63 | YES | NO | 50s | | M | 13.4 | 1.02E+08 | Fever, cancer |
| 9 | CoV-NL63 | YES | NO | 50s | | M | 13.8 | 7.71E+07 | ARI |
| 10 | CoV-NL63 | YES | NO | 10-15 | yrs | M | 19.6 | 1.38E+06 | ARI, cancer |
| 11 | CoV-NL63 | YES | NO | 60s | | F | 14 | 6.71E+07 | ARI, dehydration |
| 12 | Rhinovirus | YES | NO | 30s | | F | 27.7 | 5.04E+03 | Fever |
| 13 | Rhinovirus | YES | NO | <5 | yrs | M | 30.4 | 7.76E+02 | Altered mental status |
| 14 | Rhinovirus | YES | NO | 60s | | F | 34.5 | 4.53E+01 | Gastroenteritis |
| 15 | Rhinovirus | YES | NO | 77 | | F | 21.8 | 3.01E+05 | ARDS |
| 16 | Rhinovirus | YES | NO | 76 | | M | 18.4 | 3.18E+06 | ARI |
| 17 | Rhinovirus | YES | NO | 70s | | M | 22.9 | 1.40E+05 | ARI |
| 18 | Rhinovirus | YES | NO | 60s | | F | 24.5 | 4.63E+04 | ARI |
| 19 | Rhinovirus | YES | NO | 20s | | F | 26.2 | 1.43E+04 | ARI |
| 20 | Rhinovirus | YES | NO | 40s | | F | 27.8 | 4.71E+03 | ARI |
| 21 | Rhinovirus, bact hi | YES | High | 90s | | F | 22.1 | 2.45E+05 | Pneumonia |
| 22 | Rhinovirus, bact hi | YES | High | <5 | | F | 24 | 6.55E+04 | ARI |
| 23 | Rhinovirus, bact hi | YES | High | <5 | yrs | F | 26.1 | 1.53E+04 | ARI |
| 24 | Rhinovirus, bact hi | YES | High | 40s | | F | 30.3 | 8.32E+02 | ARI |
| 25 | Rhinovirus, bact hi | YES | High | <5 | yrs | M | 29.5 | 1.45E+03 | ARI |
| 26 | Discovered -A | YES | High | <5 | yrs | M | — | — | ARI |
| 27 | Discovered -B | no info | High | <5 | yrs | F | — | — | ARI |
| 28 | Discovered -C | no info | Detected | 50s | | M | — | — | ARI |
| 29 | Discovered -D | no info | NC | 40s | | M | — | — | ARDS |
| 30 | Discovered -E | no info | NO | 60s | | F | — | — | Fever |
| 31 | Discovered -F | no info | Detected | 70s | | F | — | — | ARI, COPD |
| 32 | Discovered -G | no info | NO | 20s | | M | — | — | Fever, dyspnea (EBV) |
| 33 | Discovered -H | no info | NO | 20s | | F | — | — | Fever, rash (CMV) |
| 34 | COVID, peak viral load | YES | n.d. | 70s | | F | 15.9 | 1.80E+07 | COVID+, moderate |
| 35 | COVID, peak viral load | YES | n.d. | 80s | | F | 11.6 | 3.54E+08 | COVID+ mild |
| 36 | COVID, peak viral load | YES | n.d. | 90s | | F | 15.5 | 2.37E+07 | COVID+, mild |
| 37 | COVID, peak viral load | YES | n.d. | 60s | | M | 15.1 | 3.13E+07 | COVID+, asymptomatic |
| 38 | COVID, peak viral load | YES | n.d. | 80s | | M | 12.8 | 1.54E+08 | COVID+, mild |
| 39 | COVID, peak viral load | YES | n.d. | 50s | | M | 17.2 | 7.30E+06 | COVID+, mild |
| 40 | COVID, peak viral load | YES | n.d. | 70s | | F | 15.5 | 2.37E+07 | COVID+, moderate |
| 41 | COVID, peak viral load | YES | n.d. | 90s | | F | 12.6 | 1.77E+08 | COVID+, mild |
| 42 | COVID, peak viral load | YES | n.d. | 50s | | M | 17.1 | 7.83E+06 | COVID+, moderate |
| 43 | COVID, peak viral load | YES | n.d. | 70s | | F | 14.6 | 4.43E+07 | COVID+, moderate |
| 44 | COVID, end of disease | YES | n.d. | 60s | | M | 37.2 | 6.96E+00 | COVID+, asymptomatic |
| | course | | | | | | | | |
| 45 | COVID, end of disease | YES | n.d. | 80s | | M | 35.1 | 2.99E+01 | COVID+, mild |
| | course | | | | | | | | |
| 46 | COVID, end of disease | YES | n.d. | 50s | | M | 36.9 | 8.57E+00 | COVID+, mild |
| | course | | | | | | | | |
| 47 | COVID, end of disease | YES | n.d. | 70s | | F | 36.7 | 9.85E+00 | COVID+, moderate |
| | course | | | | | | | | |
| 48 | COVID, end of disease | YES | n.d. | 90s | | F | 31.2 | 4.46E+02 | COVID+, mild |
| | course | | | | | | | | |
| 49 | COVID, end of disease | YES | n.d. | 50s | | M | 35.5 | 2.26E+01 | COVID+, moderate |
| | course | | | | | | | | |
| 50 | COVID, end of disease | YES | n.d. | 70s | | F | 35.4 | 2.43E+01 | COVID+, moderate |
| | course | | | | | | | | |
| 51 | COVID discovered | YES | n.d. | 60s | | M | 22 | 2.62E+05 | COVID+ no data |
| 52 | COVID discovered | YES | n.d. | <6 | months | M | 15 | 3.36E+07 | COVID+, mild |

| | | | | | | | | |
|---|---|---|---|---|---|---|---|---|
| 53 | COVID discovered | YES | n.d. | 50s | M | 27.6 | 5.40E+03 | COVID+, mild |

n.d = not done

First, cytokines were identified that were differentially expressed in virus infected and negative control subjects (FIG. 4A). Consistent with transcriptome data, these samples showed enrichment of cytokines associated with the interferon response and antiviral immunity, with CXCL10, TRAIL, IL23 being among the most associated with high viral load (FIG. 4A). COVID-19 patient samples showed distinct immunophenotypes at the peak and end of infection, with peak defined as the sample with highest viral load by RT-qPCR, and end defined as the first sample with a cycle threshold<30 for the SARS-CoV-2 N1 gene. Consistently with prior work tracking CXCL10 in these subjects (Cheemarla et al., Journal of Experimental Medicine 218 (2021)), interferon response associated cytokines were high during peak viral load, and samples towards the end of infection more closely matched the pattern seen in virus-negative controls. Notably, IL33 and TGFα were relatively depleted in COVID, end of infection samples compared to control subjects, indicating a difference between resolving infection and baseline innate immune status (FIG. 4B).

Cytokines enriched in pathobiont-samples were also examined which showed a distinct pattern, with enrichment for cytokines associated with NFKB-driven inflammation on anti-bacterial defense. Some of the sample cytokines were also identified as upstream regulators of transcripts enriched in RV positive, pathobiont high compared to RV positive, pathobiont low samples (IL1α, IL-1β, TNFα, IL6) (FIG. 4C, FIG. 6C).

### Example 7: Machine Learning Identifies Two-Cytokine Models Predictive of Viral or Pathobiont-High Immunophenotypes

Next, minimal subsets of NP biomarkers were defined that signal the presence of infection and specify infection type. To this end, machine learning was used to identify minimal cytokine combinations needed to predict virus-positive or pathobiont-high status. We previously showed that NP CXCL10 expression correlates with expression of CXCL11 and CXCL9, which also overlap in regulation and biological function as ligands for the same receptor, and all three correlate with the presence of viral infection (Landry & Foxman, The Journal of infectious diseases 217, 897-905, (2018); Groom & Luster, Exp Cell Res 317, 620-631, (2011)). However, combining two cytokines which differ in regulation and/or biological function but are both induced during viral infection might offer a bigger advantage in improving sensitivity and/or accuracy of virus detection.

To identify such pairs, Shapley analysis was used to rank the predictive value of individual cytokines for viral infection and assessed the predictive value of pairs of the top scoring cytokines in a two-feature random forest (RF) model (FIG. 7 and FIG. 4D). The pairwise combination of CXCL10 with CCL2 was the most predictive, although many cytokines and cytokine pairs performed well at distinguishing viral infection from negative controls in the proteomics sample set (FIG. 4D and Table 11). To validate the predictive value of individual and paired biomarkers, CXCL10, CCL2, and IL10 were measured, the top performing cytokines in two-feature models, in a larger, previously-described sample set (Landry & Foxman, The Journal of infectious diseases 217, 897-905, (2018)), using an automated microfluidic assay to perform cytokine measurements in triplicate, simulating what would be done for clinical use (Aldo et al., Am J Reprod Immunol 75, 678-693, (2016)). CXCL10 was the best single predictor of viral infection (sensitivity=0.91, SD 0.11; accuracy=0.88, SD 0.15), and a paired model with CXCL10 and CCL2 showed a slight increase in sensitivity and accuracy (sensitivity=0.93, SD 0.11, accuracy=0.90, SD 0.12) (FIG. 4E; Table 12).

TNF and IL8, or IL1β and IL8, were the top predictors of pathobiont-high status in paired models (FIG. 4F, Table 13), consistent with the pathobiont-associated heightened innate immunity pattern observed by transcriptomics (FIG. 3 and FIG. 6).

TABLE 11
2 feature models, accuracy and sensitivity for predicting virus positive status

| | | | | | | | | | |
|---|---|---|---|---|---|---|---|---|---|
| IL1B | IL-8 | CXCL10 | IL-15 | IP-10 | MCP-1 | CXCL9 | MCP-2 | BCA-1 | TRAIL |

2-feature models, accuracy

| | | | | | | | | | | |
|---|---|---|---|---|---|---|---|---|---|---|
| IL1B | 0.81 | 0.78 | 0.92 | 0.71 | 0.86 | 0.92 | 0.83 | 0.81 | 0.89 | 0.88 |
| IL-8 | 0.78 | 0.83 | 0.88 | 0.76 | 0.83 | 0.83 | 0.83 | 0.83 | 0.83 | 0.81 |
| CXCL10 | 0.92 | 0.88 | 0.87 | 0.83 | 0.92 | 0.89 | 0.92 | 0.87 | 0.84 | 0.94 |
| IL-15 | 0.78 | 0.83 | 0.83 | 0.71 | 0.81 | 0.81 | 0.78 | 0.76 | 0.81 | 0.86 |
| IP-10 | 0.88 | 0.83 | 0.88 | 0.87 | 0.87 | 0.94 | 0.89 | 0.87 | 0.92 | 0.87 |
| MCP-1 | 0.89 | 0.87 | 0.87 | 0.88 | 0.95 | 0.95 | 0.92 | 0.82 | 0.89 | 0.87 |
| CXCL9 | 0.88 | 0.88 | 0.92 | 0.81 | 0.89 | 0.92 | 0.76 | 0.81 | 0.92 | 0.86 |
| MCP-2 | 0.84 | 0.78 | 0.87 | 0.76 | 0.84 | 0.79 | 0.83 | 0.69 | 0.87 | 0.79 |
| BCA-1 | 0.84 | 0.89 | 0.92 | 0.88 | 0.89 | 0.89 | 0.84 | 0.87 | 0.89 | 0.93 |
| TRAIL | 0.83 | 0.83 | 0.87 | 0.87 | 0.92 | 0.84 | 0.87 | 0.78 | 0.84 | 0.78 |

STDEV, 2 feature models, accuracy

| | | | | | | | | | | |
|---|---|---|---|---|---|---|---|---|---|---|
| IL1B | 0.23 | 0.22 | 0.13 | 0.20 | 0.21 | 0.17 | 0.23 | 0.23 | 0.18 | 0.21 |
| IL-8 | 0.22 | 0.20 | 0.21 | 0.21 | 0.23 | 0.20 | 0.20 | 0.20 | 0.23 | 0.23 |
| CXCL10 | 0.13 | 0.21 | 0.14 | 0.23 | 0.13 | 0.13 | 0.13 | 0.14 | 0.17 | 0.12 |
| IL-15 | 0.22 | 0.20 | 0.23 | 0.23 | 0.20 | 0.23 | 0.22 | 0.23 | 0.20 | 0.24 |
| IP-10 | 0.21 | 0.20 | 0.21 | 0.18 | 0.18 | 0.12 | 0.13 | 0.14 | 0.13 | 0.14 |
| MCP-1 | 0.18 | 0.14 | 0.14 | 0.17 | 0.10 | 0.10 | 0.13 | 0.20 | 0.18 | 0.14 |
| CXCL9 | 0.21 | 0.21 | 0.13 | 0.23 | 0.13 | 0.13 | 0.17 | 0.23 | 0.13 | 0.21 |
| MCP-2 | 0.17 | 0.22 | 0.14 | 0.28 | 0.13 | 0.19 | 0.20 | 0.24 | 0.14 | 0.15 |
| BCA-1 | 0.17 | 0.18 | 0.13 | 0.17 | 0.13 | 0.18 | 0.17 | 0.18 | 0.18 | 0.11 |
| TRAIL | 0.20 | 0.20 | 0.14 | 0.18 | 0.13 | 0.17 | 0.14 | 0.22 | 0.17 | 0.22 |

2-feature models, sensitivity

| | | | | | | | | | | |
|---|---|---|---|---|---|---|---|---|---|---|
| IL1B | 0.98 | 0.93 | 0.98 | 0.90 | 0.98 | 1.00 | 0.98 | 0.95 | 0.98 | 0.98 |
| IL-8 | 0.93 | 0.95 | 0.98 | 0.90 | 0.95 | 0.95 | 0.95 | 0.95 | 0.95 | 0.93 |
| CXCL10 | 0.98 | 0.98 | 0.92 | 0.93 | 0.97 | 0.98 | 0.98 | 0.94 | 0.95 | 0.98 |
| IL-15 | 0.93 | 0.95 | 0.95 | 0.84 | 0.92 | 0.93 | 0.90 | 0.89 | 0.93 | 0.95 |
| IP-10 | 1.00 | 0.94 | 1.00 | 0.95 | 0.93 | 1.00 | 0.97 | 0.94 | 0.98 | 0.95 |
| MCP-1 | 1.00 | 0.95 | 0.95 | 0.93 | 1.00 | 0.98 | 1.00 | 0.89 | 0.98 | 0.95 |
| CXCL9 | 1.00 | 1.00 | 0.98 | 0.93 | 0.97 | 1.00 | 0.85 | 0.95 | 0.98 | 0.95 |
| MCP-2 | 0.92 | 0.91 | 0.94 | 0.89 | 0.94 | 0.86 | 0.98 | 0.83 | 0.94 | 0.93 |
| BCA-1 | 0.98 | 0.98 | 0.98 | 0.95 | 0.98 | 0.98 | 0.98 | 0.97 | 0.98 | 1.00 |
| TRAIL | 0.95 | 0.95 | 0.95 | 0.93 | 1.00 | 0.93 | 0.95 | 0.93 | 0.95 | 0.93 |

STDEV, 2 feature models, sensitivity

| | | | | | | | | | | |
|---|---|---|---|---|---|---|---|---|---|---|
| IL1B | 0.08 | 0.11 | 0.08 | 0.17 | 0.08 | 0.00 | 0.08 | 0.15 | 0.08 | 0.08 |
| IL-8 | 0.11 | 0.10 | 0.08 | 0.17 | 0.15 | 0.10 | 0.10 | 0.10 | 0.10 | 0.16 |
| CXCL10 | 0.08 | 0.08 | 0.13 | 0.16 | 0.10 | 0.08 | 0.08 | 0.12 | 0.10 | 0.08 |
| IL-15 | 0.16 | 0.10 | 0.15 | 0.23 | 0.13 | 0.16 | 0.17 | 0.18 | 0.11 | 0.15 |
| IP-10 | 0.00 | 0.12 | 0.00 | 0.15 | 0.20 | 0.00 | 0.10 | 0.12 | 0.08 | 0.10 |
| MCP-1 | 0.00 | 0.10 | 0.10 | 0.16 | 0.00 | 0.08 | 0.00 | 0.18 | 0.08 | 0.10 |
| CXCL9 | 0.00 | 0.00 | 0.08 | 0.16 | 0.10 | 0.00 | 0.17 | 0.15 | 0.08 | 0.10 |
| MCP-2 | 0.17 | 0.14 | 0.12 | 0.24 | 0.12 | 0.18 | 0.08 | 0.24 | 0.12 | 0.16 |
| BCA-1 | 0.08 | 0.08 | 0.08 | 0.15 | 0.08 | 0.08 | 0.08 | 0.10 | 0.08 | 0.00 |
| TRAIL | 0.10 | 0.10 | 0.10 | 0.16 | 0.00 | 0.16 | 0.10 | 0.16 | 0.15 | 0.16 |

TABLE 12
Accuracies, sensitivities, and S.D. for 2-feature random forest models for
predicting “virus positive” in validation data set.

| feat1 | feat2 | Accuracy | SD |
|---|---|---|---|
| CXCL10 | CXCL10 | 0.878571429 | 0.146945468 |
| MCP1 | CXCL10 | 0.895238095 | 0.119375052 |
| IL10 | CXCL10 | 0.864285714 | 0.157820072 |
| CXCL10 | MCP1 | 0.895238095 | 0.119375052 |
| MCP1 | MCP1 | 0.688095238 | 0.127974806 |
| IL10 | MCP1 | 0.66547619 | 0.169341245 |
| CXCL10 | IL10 | 0.864285714 | 0.157820072 |
| MCP1 | IL10 | 0.66547619 | 0.169341245 |
| IL10 | IL10 | 0.65952381 | 0.118594375 |
| feat1 | feat2 | Sensitivity | SD |
| CXCL10 | CXCL10 | 0.908333333 | 0.114564392 |
| MCP1 | CXCL10 | 0.929166667 | 0.107716224 |
| IL10 | CXCL10 | 0.891666667 | 0.166666667 |
| CXCL10 | MCP1 | 0.929166667 | 0.107716224 |
| MCP1 | MCP1 | 0.793333333 | 0.196383978 |
| IL10 | MCP1 | 0.755833333 | 0.159820416 |
| CXCL10 | IL10 | 0.891666667 | 0.166666667 |
| MCP1 | IL10 | 0.755833333 | 0.159820416 |
| IL10 | IL10 | 0.71 | 0.194393644 |

TABLE 13
Accuracies and S.D. for 2-feature random forest models for predicting
“pathobiont high” in proteomics data set.

| feat1 | feat2 | acc.c | sd.c |
|---|---|---|---|
| TNF | IL-8 | 0.933333333 | 0.160669034 |
| IL-8 | TNF | 0.933333333 | 0.160669034 |
| IL-8 | IL1B | 0.85 | 0.189296945 |
| IL1B | IL-8 | 0.85 | 0.189296945 |
| RANTES | G-CSF | 0.829166667 | 0.185902551 |
| G-CSF | RANTES | 0.829166667 | 0.185902551 |
| CCL3 | IL-8 | 0.820833333 | 0.259830715 |
| IL-8 | CCL3 | 0.820833333 | 0.259830715 |
| IL1B | IL1B | 0.816666667 | 0.213437475 |
| RANTES | IL-8 | 0.816666667 | 0.240947205 |
| IL-8 | RANTES | 0.816666667 | 0.240947205 |
| IL-1RA | IL1B | 0.8125 | 0.184973047 |
| IL1B | IL-1RA | 0.8125 | 0.184973047 |
| IL-8 | G-CSF | 0.8 | 0.186592917 |
| IL-12p40 | IL1B | 0.8 | 0.232102396 |
| G-CSF | IL-8 | 0.8 | 0.186592917 |
| IL-10 [symbol] | IL-12p40 | 0.8 | 0.232102396 |
| CCL4 | IL-8 | 0.8 | 0.232805311 |
| IL-8 | CCL4 | 0.8 | 0.232805311 |
| IL-8 | IL-8 | 0.791666667 | 0.239356777 |
| CCL3 | CCL3 | 0.791666667 | 0.233482095 |
| CCL4 | IL1B | 0.7875 | 0.220841709 |
| CCL3 | IL-1RA | 0.7875 | 0.161492574 |
| IL-1RA | CCL3 | 0.7875 | 0.161492574 |
| IL1B | CCL4 | 0.7875 | 0.220841709 |
| G-CSF | G-CSF | 0.783333333 | 0.2081666 |
| IL1B | G-CSF | 0.783333333 | 0.2081666 |
| G-CSF | IL1B | 0.783333333 | 0.2081666 |
| CCL3 | IL1B | 0.783333333 | 0.2081666 |
| IL-8 | IL-1RA | 0.783333333 | 0.221175436 |
| IL-1RA | IL-8 | 0.783333333 | 0.221175436 |
| IL1B | CCL3 | 0.783333333 | 0.2081666 |
| CCL3 | G-CSF | 0.779166667 | 0.231279458 |
| G-CSF | CCL3 | 0.779166667 | 0.231279458 |
| CCL3 | Eotaxin | 0.7625 | 0.183496178 |
| IL-12p40 | IL-8 | 0.7625 | 0.228862416 |
| IL-8 | IL-12p40 | 0.7625 | 0.228862416 |
| Eotaxin | CCL3 | 0.7625 | 0.183496178 |
| CCL4 | IL-1RA | 0.758333333 | 0.190333199 |
| IL-1RA | CCL4 | 0.758333333 | 0.190333199 |
| TNF | RANTES | 0.758333333 | 0.196452096 |
| RANTES | TNF | 0.758333333 | 0.196452096 |
| CCL4 | G-CSF | 0.754166667 | 0.236120669 |
| G-CSF | CCL4 | 0.754166667 | 0.236120669 |
| TNF [symbol] | IL-1RA | 0.75 | 0.219301585 |
| IL-1RA | TNF | 0.75 | 0.219301585 |
| IL1B | Eotaxin | 0.745833333 | 0.230647515 |
| IL-12p40 | G-CSF | 0.745833333 | 0.266277523 |
| Eotaxin | IL1B | 0.745833333 | 0.230647515 |
| G-CSF | IL-12p40 | 0.745833333 | 0.266277523 |
| TNF [symbol] | IL1B | 0.741666667 | 0.218269352 |
| IL-1RA | IL-1RA | 0.741666667 | 0.248467525 |
| IL1B | TNF | 0.741666667 | 0.218269352 |
| TNF | TNF | 0.733333333 | 0.185592145 |
| TNF [symbol] | G-CSF | 0.729166667 | 0.248207835 |
| G-CSF | TNF | 0.729166667 | 0.248207835 |
| RANTES | IL1B | 0.720833333 | 0.211247081 |
| IL-1 [symbol] | RANTES | 0.720833333 | 0.211247081 |
| IL-1RA | G-CSF | 0.708333333 | 0.250277778 |
| G-CSF | IL-1RA | 0.708333333 | 0.250277778 |
| IL-8 | Eotaxin | 0.7 | 0.245101283 |
| Eotaxin | IL-8 | 0.7 | 0.245101283 |
| TNF | CCL4 | 0.7 | 0.285884508 |
| CCL4 | TNF | 0.7 | 0.285884508 |
| RANTES | Eotaxin | 0.691666667 | 0.240894975 |
| CCL3 | IL-12p40 | 0.691666667 | 0.263252107 |
| IL-12p40 | CCL3 | 0.691666667 | 0.263252107 |
| Eotaxin | RANTES | 0.691666667 | 0.240894975 |
| TNF [symbol] | CCL3 | 0.683333333 | 0.209433267 |
| CCL3 | TNF | 0.683333333 | 0.209433267 |
| TNF | Eotaxin | 0.679166667 | 0.220358227 |
| Eotaxin | TNF | 0.679166667 | 0.220358227 |
| RANTES | IL-12p40 | 0.675 | 0.273008829 |
| IL-12p40 | RANTES | 0.675 | 0.273008829 |
| CCL4 | CCL3 | 0.675 | 0.253464279 |
| CCL3 | CCL4 | 0.675 | 0.253464279 |
| RANTES | CCL3 | 0.6625 | 0.177606288 |
| CCL3 | RANTES | 0.6625 | 0.177606288 |
| IL-12p40 | IL-1RA | 0.658333333 | 0.218418816 |
| IL-1RA | IL-12p40 | 0.658333333 | 0.218418816 |
| CCL4 | CCL4 | 0.658333333 | 0.264706336 |
| CCL4 | Eotaxin | 0.645833333 | 0.260743163 |
| Eotaxin | CCL4 | 0.645833333 | 0.260743163 |
| RANTES | CCL4 | 0.645833333 | 0.263369857 |
| CCL4 | RANTES | 0.645833333 | 0.263369857 |
| G-CSF | Eotaxin | 0.641666667 | 0.226158022 |
| Eotaxin | G-CSF | 0.641666667 | 0.226158022 |
| Eotaxin | Eotaxin | 0.633333333 | 0.197905701 |
| CCL4 | IL-12p40 | 0.625 | 0.281669492 |
| IL-12p40 | CCL4 | 0.625 | 0.281669492 |
| IL-1RA | Eotaxin | 0.616666667 | 0.2961207 |
| Eotaxin | IL-1RA | 0.616666667 | 0.2961207 |
| IL-12p40 | Eotaxin | 0.608333333 | 0.292514404 |
| Eotaxin | IL-12p40 | 0.608333333 | 0.292514404 |
| TNF [symbol] | IL-12p40 | 0.604166667 | 0.241108041 |
| IL-12p40 | TNF | 0.604166667 | 0.241108041 |
| RANTES | RANTES | 0.566666667 | 0.2 |
| RANTES | IL-1RA | 0.545833333 | 0.25222767 |
| IL-1RA | RANTES | 0.545833333 | 0.25222767 |
| IL-12p40 | IL-12p40 | 0.541666667 | 0.227455734 |

### Example 8: 6-Cytokine Signature Sorts NP Samples into Infection-Associated Immunophenotypes

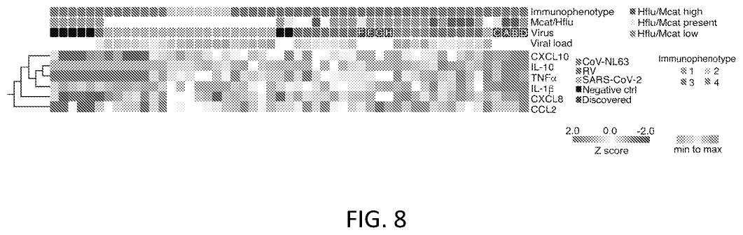

To visualize whether a minimal set of cytokines could identify infection-relevant NP immunophenotypes, samples were clustered using the top cytokine predictors of virus-positive or pathobiont-associated status in machine learning models (CXCL10, CCL2, IL10, IL8, TNF, and IL1β; FIG. 4D, 4F). UMAP plots based on 6 cytokine levels recapitulated many aspects of the UMAP plots based on >2000 differentially expressed genes (FIG. 4G-4I; FIG. 3B-3D). Samples segregated into four major clusters: (1) negative control and COVID-19, end of infection, (2) COVID-19, peak of infection, (3) virus-positive including some COVID-19 peak, (4), pathobiont-high/heightened innate immunity (FIG. 4H). Within the same cluster, cytokine levels correlated loosely with viral load (FIG. 8). COVID-19, peak samples in cluster 2 had lower overall cytokine levels than COVID-19, peak samples in cluster 3 (FIG. 8) As with transcriptome-based clustering, we saw a correlation between heightened innate immunity, pathobiont high status, and young age.

Cytokine signatures of screen-positive samples were also reminiscent of the immunophenotype patterns seen by transcriptomics. For example, the ICV- and H. influenzae-positive sample (A), another pathobiont-high sample (B), and an H. influenzae-detected sample (C), mapped to the heightened innate immunity cluster. The SARS-CoV-2 positive samples discovered in the 2020 screen fell into two clusters. One sample, from a febrile infant who was seen as an outpatient, fell within the heightened innate immunity cluster, consistent with a correlation between this immunophenotype and young age. For this sample, there was not sufficient nucleic acid for sequencing, so pathobiont status could not be ascertained. Other SARS-CoV-2-positive discovered samples were from adults and clustered with the COVID-19, end and negative control samples, possibly indicating that these samples were collected relatively late in the disease course. Overall, comparing immunophenotypes based on transcriptome (FIG. 3) and proteome (FIG. 4) shows that a 6-cytokine signature can identify infection-associated NP immunophenotypes, and shows promise for distinguishing viral ARI from ARI associated with bacterial pathobionts.

In sum, these results show that measuring one or a few cytokines in NP swab samples can enrich for samples likely to contain respiratory pathogens, and to some degree can categorize infection type.

The disclosure presents an efficient pathogen surveillance strategy using respiratory swabs from symptomatic patients that have tested negative on a standard diagnostic respiratory virus panel.

The problem to be solved is that cough, fatigue, and other symptoms which lead to respiratory virus testing have many possible non-infectious causes, making it inefficient and cost-prohibitive to search for undiagnosed pathogens in every patient. The disclosure demonstrates that screening for a single cytokine in NP samples identifies a fraction of the total samples (<10%, FIGS. 1B and 2C) as those most likely to contain undiagnosed infections. The discovery of 2- and 6-cytokine signatures that better parse infection-associated NP immunophenotypes was unexpected and suggests that adding a few more biomarkers could further improve the efficiency this strategy (FIG. 4).

Host response-based screening is an attractive strategy for surveillance for emerging viruses. Since this approach relies on immune recognition of features common to many viruses, this requires no prior knowledge of the pathogen. Host response-based approaches could also be used to identify zoonotic pathogens, as illustrated by a recent study in which a novel picomavirus was discovered in Zebrafish after investigators noticed an interferon signature (Balla et al., Current biology: CB 30, 2092-2103 e2095, (2020)). Blood biomarkers of the interferon response can also be used to signal viral respiratory infection and could potentially be used for pathogen discovery (McClain et al., Lancet Infect Dis 21, 396-404, (2021); Gupta et al., Lancet Microbe 2, e508-e517, (2021)). However, NP samples offer the advantage that the pathogen can be identified, sequenced, and even cultured directly from the same sample used for screening (FIG. 1D, FIG. 2D, FIG. 5).

When using NP biomarkers to detect undiagnosed infections, it is important to consider that the innate immune response is dynamic and may be less robust as viral load declines, as evident from the clustering of SARS-CoV-2 positive NP swabs into distinct immunophenotypes depending on whether they are from the peak or the end of infection (FIGS. 4B and 4G, FIG. 8) (Cheemarla et al., Journal of Experimental Medicine 218 (2021). However, although host-response based screening may not capture every viral infection, particularly when the viral load is low, robust host response generally correlates with higher viral load, based on our observation in this study and prior studies (FIG. 4A-4C, FIG. 8, and prior studies ((Landry & Foxman, The Journal of infectious diseases 217, 897-905, (2018); Cheemarla et al., Journal of Experimental Medicine 218 (2021)). This is also an advantage in finding high-yield specimens for virus discovery, in that samples identified by innate immune response biomarkers are also more likely to have a high enough viral load to enable downstream analyses such as viral culture, isolation, and sequencing.

While the focus of this study was detecting undiagnosed pathogens, our results from deep characterization of NP immunophenotypes and identification of biomarker signatures by machine learning suggests additional uses for host-response based testing in patient care. For example, in patients with ARI, we observed an interferon response pattern associated with viral respiratory infection, and a distinct heightened innate immunity phenotype correlating with leukocyte infiltration and high levels of bacterial pathobionts H. influenzae or M. catarrhalis (FIG. 3 and FIG. 4). Given that outpatient ARI is the most common condition linked to antibiotic overuse (Fleming-Dutra et al., JAMA 315, 1864-1873), NP biomarkers of these immunophenotypes could be useful in distinguishing viral-only ARI from ARI associated with pathogenic bacteria, enabling more informed decisions about whether patients would benefit from antibiotics. We also found three instances of elevated NP CXCL10 in patients with acute CMV or EBV (Table 3), suggesting that NP CXCL10 may be a biomarker for these infections, which are typically diagnosed by serology. Additional work characterizing pathogens and NP host response in a wider variety of samples will be needed to determine how generalizable these patterns are. However, the general approach of using deep characterization followed by machine learning to identify minimal biomarker signatures of disease-relevant NP immunophenotypes provides a framework for how diagnostically useful NP biomarkers can be defined.

The disclosure also reveals some interesting facets of host-pathogen interactions in the nasopharynx that merit further study. The metatranscriptomic and proteomic data suggest a previously unrecognized association between heightened NP innate responses, high levels of bacterial pathobionts, and young age (FIG. 3B, FIG. 4G-4I). Two recent studies demonstrated heightened NP innate immunity in children compared to adults with COVID-19, but these studies did not assess NP bacteria (Loske et al., Nat Biotechnol, (2021); Pierce et al., JCI Insight 6 (2021)). Interestingly, a recent report shows that young age is associated with lower production of antibacterial proteins by the airway mucosa, increasing susceptibility to bacterial colonization (Lokken-Toyli et al., Mucosal Immunol, 14(6):1358-1368 (2021)); also, a prior study showed that H. influenzae or M. catarrhalis can drive the expression of IL10 and TNF in the airway mucosa of asymptomatic infants, reminiscent of the heightened innate immunity phenotype that was observed (Folsgaard et al., Am J Respir Crit Care Med 187, 589-595 (2013)). Together, these data suggest that bacterial pathobionts H. influenzae or M. catarrhalis may be the drivers, or among the drivers, of the heightened NP innate immunity described in children, and compel further studies of the relationship between age, mucosal bacteria, and antiviral defense.

In conclusion, here we show that measuring one or several cytokines in patient nasopharyngeal samples can enrich for patient samples containing missed infections, allowing efficient use of patient samples for pathogen discovery, and augmenting current strategies for infectious disease diagnosis and surveillance.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for detecting and distinguishing between a viral-only or a bacterial-associated respiratory infection in a patient, the method comprising:
analyzing a respiratory sample to determine levels of at least two respiratory virus infection-associated molecules, at least two bacterial respiratory infection-associated molecules, or a combination thereof;
comparing the levels of the respiratory virus infection-associated molecules and/or the levels of the bacterial respiratory infection-associated molecules with a predetermined reference level for the respiratory virus infection-associated molecules and/or a predetermined reference level for the bacterial respiratory infection-associated molecules; and
determining if the patient has a virus-associated respiratory infection or a bacterial-associated respiratory infection based upon the comparing of the levels.

2. The method of claim 1, wherein the at least two respiratory virus infection-associated molecules are selected from the group comprising CXCL10, TRAIL, IL-23, CCL2, IL-10, IL-6, CCL15, TNFα, M-CSF, CX3CL1, CXCL9, IL-15, CCL22, IL-16, G-CSF, IL-1α, IL-8, CCL8, BCA1, IL-1β, IFNγ, CCL17, IL-12p40, sCD40L, and CCL27.

3. The method of claim 1, wherein the at least two respiratory virus infection-associated molecules are selected from the group comprising CXCL10, TRAIL, IL-23, CCL2, IL-10, IL-6, CCL15, M-CSF, CX3CL1, CXCL9, IL-15, CCL22, IL-16, IL-1α, CCL8, BCA1, IFNγ, CCL17, sCD40L, and CCL27.

4. The method of claim 1, wherein the at least two respiratory virus infection-associated molecules are selected from the group comprising BCA1, IL-15, IL-10, CCL8, CCL2, CXCL10, CXCL9, TRAIL, IL-8, IL-1β, IFNγ, CCL17, IL-12p40, sCD40L, M-CSF, and/or CCL27.

5. The method of claim 1, wherein the at least two respiratory virus infection-associated molecules are selected from the group comprising CCL8, IL-15, CXC13, IL-10, CCL2, CXCL10, TRAIL, CXCL9, IL-13 and IL-8.

6. The method of claim 1, wherein the at least two respiratory virus infection-associated molecules are selected from the group comprising CXCL10, CCL2, and IL-10.

7. The method of claim 1, wherein the at least two respiratory virus infection-associated molecules include CXCL10 and CCL2.

8. The method of claim 1, wherein the at least two bacterial respiratory infection-associated molecules are selected from the group comprising CCL5, IL-1RA, CCL11, IL-12p40, CCL3, G-CSF, IL-8, TNFα, IL-1β, CCL4, IL-1α, IL-22, IL-6, RANTES, MIP-1β, MIP-1α, Eotaxin, GROα, CCL27, MCP-3, SCF, IL-13, IL-16, IL-10, EGF, CCL17, CXCL9, and FGF-2.

9. The method of claim 1, wherein the at least two bacterial respiratory infection-associated molecules are selected from the group comprising CCL5, IL-1RA, CCL11, IL-12p40, CCL3, G-CSF, IL-8, TNFα, IL-1β, CCL4, IL-1α, IL-22, and IL-6.

10. The method of claim 1, wherein the at least two bacterial respiratory infection-associated molecules are selected from the group comprising CCL5, IL-1RA, CCL11, IL-12p40, CCL3, G-CSF, IL-8, TNFα, IL-1β, and CCL4.

11. The method of claim 1, wherein the at least two bacterial respiratory infection-associated molecules are selected from the group comprising TNF, IL-8, and IL-1β.

12. The method according to claim 1, wherein analyzing a respiratory sample comprises determining levels of CXCL10, CCL2, IL-10, IL-8, TNF, and IL-1β.

13. The method of claim 1, wherein the expression level of the respiratory virus infection-associated molecules or the bacterial respiratory infection-associated molecules is determined by measuring the protein level of the molecule.

14. The method of claim 13, wherein the protein level is determined by ELISA, an immunoassay, or mass spectrometry.

15. The method according to claim 1, wherein determining that the patient has a virus-associated respiratory infection includes determining that the levels of the respiratory virus infection-associated molecules are above the respective reference level.

16. The method according to claim 15, further comprising the step of treating the patient with antiviral drugs.

17. The method according to claim 1, wherein determining that the patient has a bacterial-associated respiratory infection includes determining that the levels of the bacterial respiratory infection-associated molecules are above the respective reference level.

18. The method according to claim 17, further comprising the step of treating the patient with antibiotics.

19. A method of determining whether a subject who tests positive for the presence of bacterial or viral respiratory pathogen is a carrier or if the pathogen is part of the disease process, the method comprising:
a. analyzing a respiratory sample to determine a level of at least one respiratory virus infection-associated molecule and a level of at least one bacterial respiratory infection-associated molecule; and
b. comparing the level of the at least one respiratory virus infection-associated molecule and the level of the at least one bacterial respiratory infection-associated molecule with a predetermined reference level for the at least one respiratory virus infection-associated molecule and a predetermined reference level of the at least one bacterial respiratory infection-associated molecule;
wherein:
if the level of the at least one respiratory virus infection-associated molecule is above the respective reference level, the patient is determined to have a respiratory viral infection;
if the level of the at least one bacterial respiratory infection-associated molecule is above the respective reference level, the patient is determined to have a bacterial-associated respiratory infection;
if the level of the at least one respiratory virus infection-associated molecule is above the respective reference level and the level of the at least one bacterial respiratory infection-associated molecule is above the respective reference level, the patient is determined to have both a respiratory viral infection and a bacterial respiratory infection; or
if neither the level of the at least one respiratory virus infection-associated molecule nor the level of the at least one bacterial respiratory infection-associated molecule is above the respective reference level, the subject is determined to be a carrier.

20. A method for excluding the presence of a coronavirus in a sample from a patient, the method comprising:
a. analyzing a respiratory sample to determine an expression level of at least one respiratory virus infection-associated molecule; and
b. comparing the level of the at least one respiratory virus infection-associated molecule with a predetermined reference level for the at least one respiratory virus infection-associated molecule;
wherein if the level of the at least one respiratory virus infection-associated molecule is below the respective reference level, the presence of a coronavirus is excluded.
